(19) United States
(12) Patent Application Publication
(10) Pub. No.: US 2004/0152156 A1
(43) Pub. Date: Aug. 5, 2004

(54) Regulation of human b7-h2 protein

(76) Inventors: Jeffrey Encinas, Kyoto-shi, Kyoto-fu (JP); Eri Tanabe, Nara-shi Nara-ken (JP); Shinichi Watanabe, Nara-shi Nara-ken (JP)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/250,533

(22) PCT Filed: Jan. 4, 2002
(86) PCT No.: PCT/EP02/00028

(51) Int. Cl. C07K 14/47; C07H 21/04

(52) U.S. Cl. 435/69.1; 435/320.1; 435/325; 530/350; 536/23.5

(57) ABSTRACT

Reagents which regulate human B7-H2 and reagents which bind to human B7-H2 gene products can play a role in preventing, ameliorating, or correcting dysfunctions or diseases including, but not limited to, allergic diseases, such as respiratory allergies, food allergies, asthma, and atopic dermatitis, as well as in the treatment of intracellular bacterial infections, such as tuberculosis, leprosy, listeriosis, and salmonellosis; and autoimmune diseases, such as multiple sclerosis, rheumatoid arthritis, and type I diabetes, as well as in the treatment of helminth and extracellular microbial infections.

### TECHNICAL FIELD OF THE INVENTION

The invention relates to nucleotide and amino acid sequences of human B7-H2 and to the regulation of the same.

### BACKGROUND OF THE INVENTION

B7 family ligands, expressed on antigen presenting cells, are the counter-ligands for several receptors expressed on T lymphocytes. Costimulatory interactions between the B7 family ligands and their receptors play critical roles in the growth, differentiation, and death of T cells. Engagement of the T-cell costimulator CD28, constitutively expressed on resting T cells, by its natural ligands B7-1 and B7-2 increases antigen-specific proliferation of CD4⁺ T cells, enhances production of cytokines, induces maturation of CD8+ effector T cells (Chambers C A, Allison J P. (1997) Co-stimulation in T cell responses. Curr Opin Immunol., 9, 396-404; Lenschow D J et al (1996) Bluestone J A. C28/B7 system of T cell costimulation. Annu Rev Immunol. 14, 233-258; Chen L, Linsley P S, Hellstrom K E (1993) Costimulation of T cells for tumor immunity. Immnol Today. 14, 483-486), and promotes T-cell survival (Boise L H, Noel P J, Thompson C B. CD28 and apoptosis. (1995) Curr Opin Immunol., 7, 620-625). Another ligand, termed CTLA4 is homologous to CD28 but is not expressed on resting T cells and appears following T cell activation (Brunet, J. F., et al., (1987) Nature 328, 267-270). Signaling through homologous CTLA-4 receptor of B7-1 and B7-2 on activated T cells is thought to deliver a negative signal that inhibits T-cell proliferation, IL-2 production, and cell cycle progression (Krummel M F, Allison J P. (1996) CTLA4 engagement inhibits IL-2 accumulation and cell cycle progression upon activation of resting T cells. J Exp Med., 183, 2533-2540; Walunas T L, Bakker C Y, Bluestone J A. (1996) CTLA-4 ligation blocks CD28-dependent T cell activation. J Exp Med. 183, 2541-2550).

Therefore, manipulation of the B7:CD28/CTLA4 pathway offers great potential to stimulate or suppress immune responses in humans.

Recent studies indicate other new members of the B7-CD28 family may also participate in the regulation of cellular and humoral immune responses. One of the new members is B7-like gene designated B7-H1 (B7 homolog 1) and B7-H2 (B7 homolog 2). B7-H2 binds an inducible costimulator (ICOS), a homolog of the B7-1 and B7-2 receptors CD28 and CTLA-4 (CD152).

The transcript of the B7-H2 gene was originally described by the Kazusa DNA institute as a cDNA clone derived from Homo sapiens adult male brain (ref 1). Recently, however, owing to the homology between B7-H2 and the costimulatory molecules B7-1 (CD80) and B7-2 (CD86), it was found that B7-H2 is a ligand for ICOS, a homolog of the B7-1 and B7-2 receptors CD28 and CTLA-4 (CD152) (refs. 2-7).

ICOS is a costimulatory receptor whose expression is upregulated on CD4+ and CD8⁺ T cells after T cell receptor stimulation (refs. 8-10). Stimulation of ICOS is thought to induce the production of IL-10 cytokine production, and to a lesser extent to increase production of IL-4, IL-5, IFN-γ, TNF-α, and GM-CSF, as well as to promote the function of activated Th2 helper cells (refs. 9, 10). The ICOS gene has been reported to be expressed predominantly in primary and secondary lymphoid tissues (ref 3)

There is a need in the art to identify novel variants of B7-H2 proteins which can be regulated and provide therapeutic options.

### SUMMARY OF TH INVENTION

It is an object of the invention to provide novel polynucleotides encoding novel polypeptides of B7-H2 splice variants (137-H2 V), or biologically active derivatives thereof. The polynucleotides of the present invention have the polynucleotide sequence selected from the group consisting of the sequence as depicted in SEQ ID NO:1, the polynucleotide sequence which hybridizes to the sequence as depicted in SEQ ID NO:1 under the stringent condition, the sequence as depicted in SEQ ID NO:2, and the polynucleotide sequence which hybridizes to the sequence as depicted in SEQ ID NO:2 under the stringent condition.

The polypeptide of the present invention comprises the amino acid sequence selected from the group consisting of the amino acid sequence as depicted in SEQ ID NO:3, amino acid sequences wherein a substitution, deletion, addition or transposition of one to several amino acid residue(s) is made in SEQ ID NO:3, the amino acid sequence as depicted in SEQ ID NO:4, amino acid sequences wherein a substitution, deletion, addition, or transposition of one to several amino acid residue (s) is made in SEQ ID NO:4.

It is also an object of the present invention to provide reagents and methods of regulating a human B7-H2. This and other objects of the invention are provided by one or more of the embodiments described below.

One embodiment of the invention is a method of screening for agents which can regulate the activity of a human B7-H2. A test compound is contacted with a polypeptide comprising an amino acid sequence which is at least about 90% identical to the amino acid sequence shown in SEQ ID NO: 3 or 4. Binding of the test compound to the polypeptide is detected. A test compound which binds to the polypeptide is thereby identified as a potential therapeutic agent for regulating activity of the human B7-H2.

Another embodiment of the invention is a method of screening for agents which regulate an activity of a human B7-H2. A test compound is contacted with a polypeptide comprising an amino acid sequence which is at least about 90% identical to the amino acid sequence shown in SEQ ID NO:3 or 4. A B7-H2 like activity of the polypeptide is detected. A test compound that decreases the B7-H2 like activity is thereby identified as a potential therapeutic agent for decreasing the activity of the human B7-H2. A test compound which increases the B7-H2 like activity of the polypeptide is thereby identified as a potential therapeutic agent for increasing the activity of the human B7-H2.

Yet another embodiment of the invention is a method of screening for agents which regulate an activity of a human B7-H2. A test compound is contacted with a product encoded by a polynucleotide which comprises a nucleotide sequence which is at least 90% identical to the nucleotide sequence shown in SEQ ID NO:1 or 2. Binding of the test compound to the product is detected. A test compound which binds to the product is thereby identified as a potential therapeutic agent for regulating the activity of the human B7-H2.

Even another embodiment of the invention is a method of reducing activity of a human B7-H2. A cell is contacted with a reagent which specifically binds to a product encoded by a polynucleotide comprising a nucleotide sequence which is at least 90% identical to the nucleotide sequence shown in SEQ ID NO:1 or 2. The activity of the human B7-H2 is thereby reduced.

Another embodiment of the invention is a pharmaceutical composition comprising a reagent which specifically binds to a product encoded by a polynucleotide comprising a nucleotide sequence which is at least 90% identical to the nucleotide sequence shown in SEQ ID NO:1 or 2 and a pharmaceutically acceptable carrier.

Another embodiment of the invention is a pharmaceutical composition comprising an expression construct encoding a polypeptide comprising the amino acid sequence shown in SEQ ID NO:3 or 4 and a pharmaceutically acceptable carrier.

Yet another embodiment of the invention is an isolated and purified polynucleotide consisting essentially of the nucleotide sequence shown in SEQ ID NO:1 or 2.

Still another embodiment of the invention is an isolated and purified polypeptide consisting essentially of the amino acid sequence shown in SEQ ID NO:3 or 4.

Even another embodiment of the invention is a preparation of antibodies which specifically binds to a polypeptide consisting essentially of the amino acid sequence shown in SEQ ID NO:3 or 4.

A further embodiment of the invention is a method of preparing a polypeptide consisting essentially of the amino acid sequence shown in SEQ ID NO:3 or 4. A host cell comprising an expression construct encoding the polypeptide is cultured under conditions whereby the polypeptide is expressed. The polypeptide is isolated.

The invention thus provides a human B7-H2 which can be used to identify test compounds which may act, for example, as enhancers or inhibitors of formation of the receptor complex. Human B7-H2 and fragments thereof also are useful in raising specific antibodies which can block the protein and effectively reduce its activity.

### BRIEF DESCRIPTION OF THE DRAWINGS

FIG. 1. shows the alignment of human B7-H2 alternative splice variants, nucleotide sequences (SEQ ID NO:1 or SEQ ID NO:2) of the present invention with other variants of B7-H2.

FIG. 2. shows the alignment of human B7-H2 alternative splice variants, amino acid sequence (SEQ ID NO:3 or SEQ ID NO:4) of the present invention against other variants of B7-H2.

FIG. 3. shows the expression profiling of B7-H2 transcript 1 or 2 mRNA.

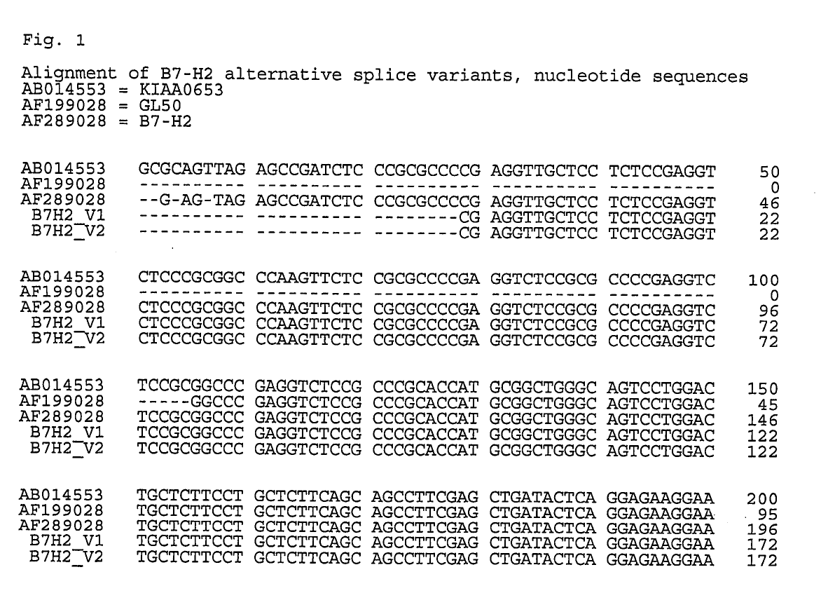

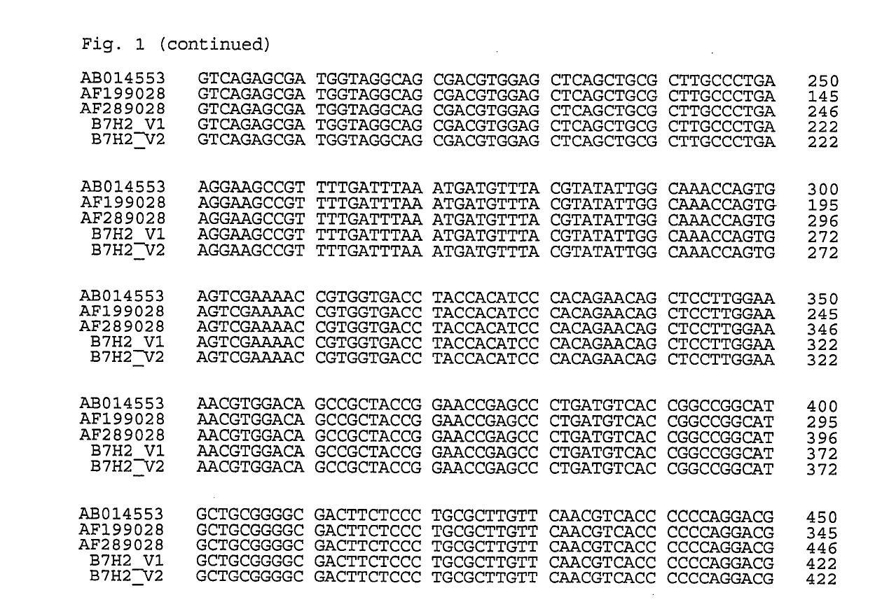

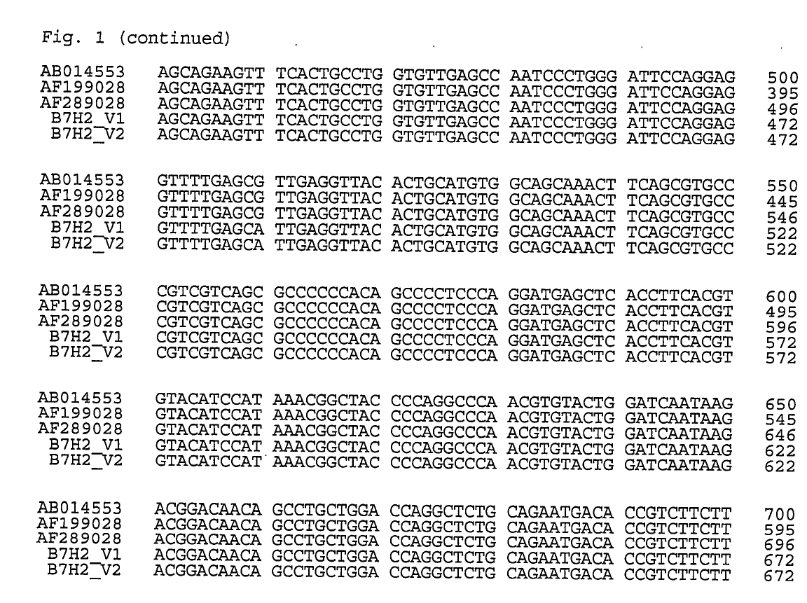

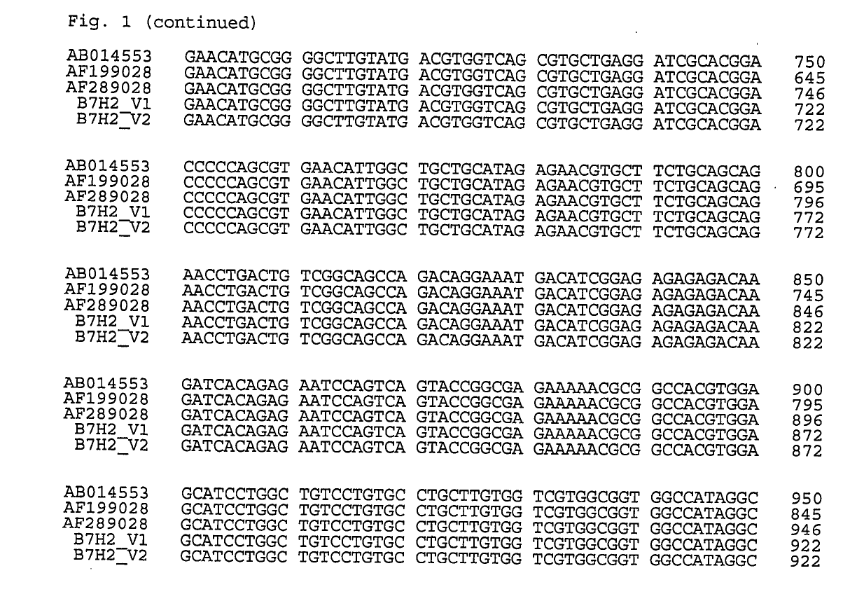

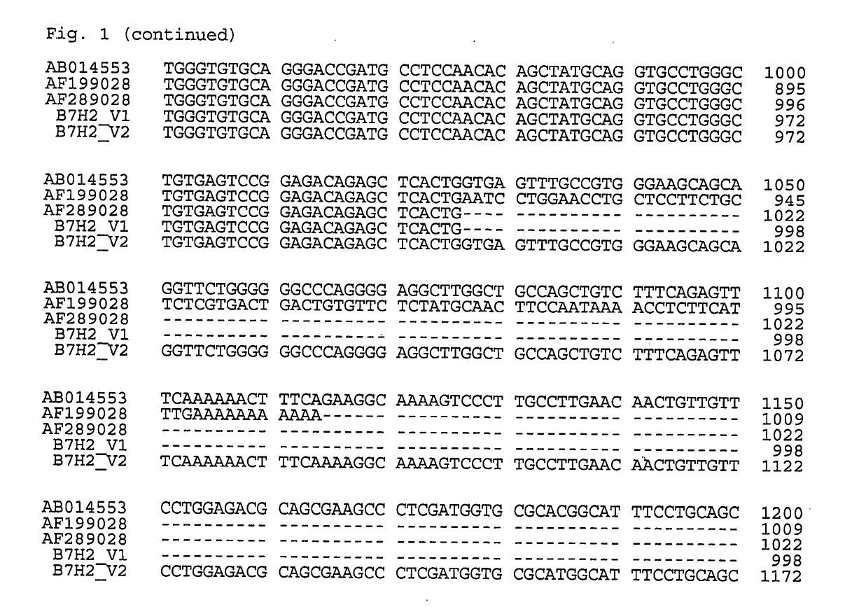

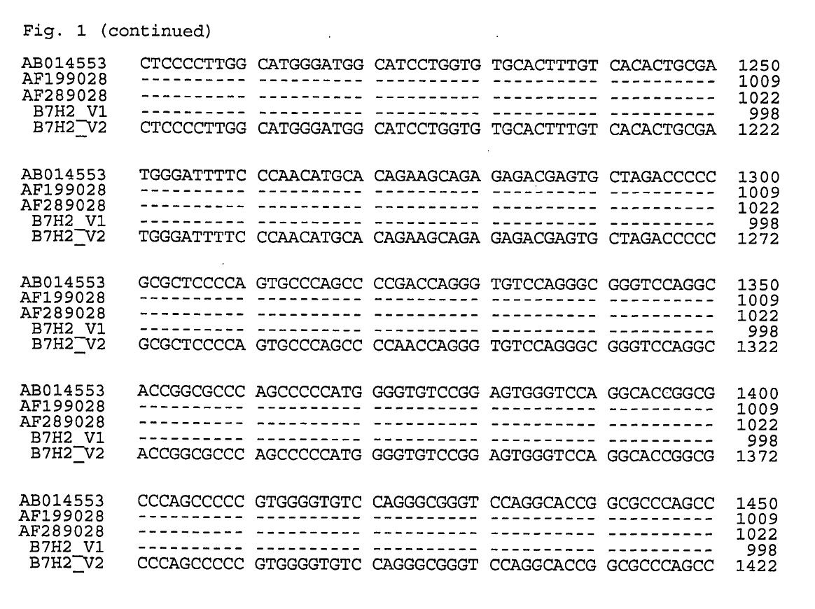

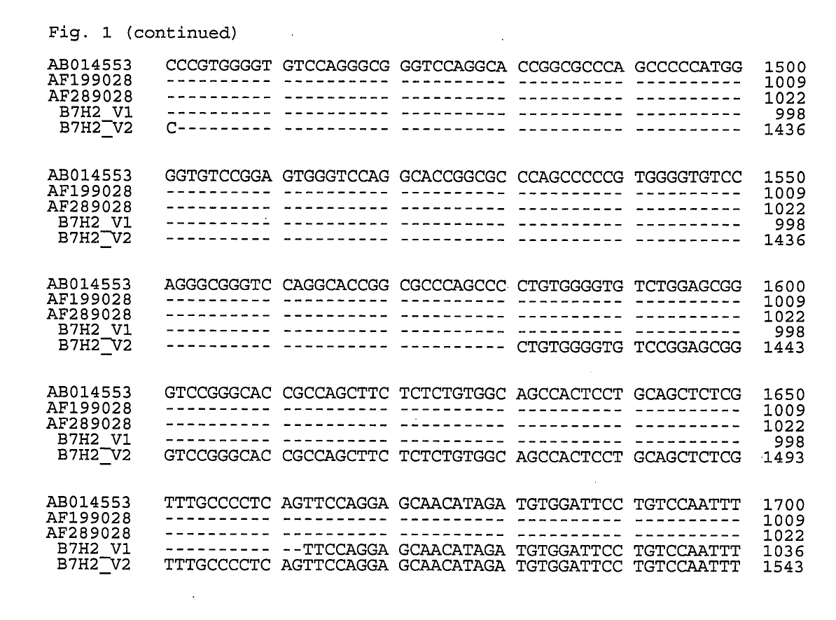

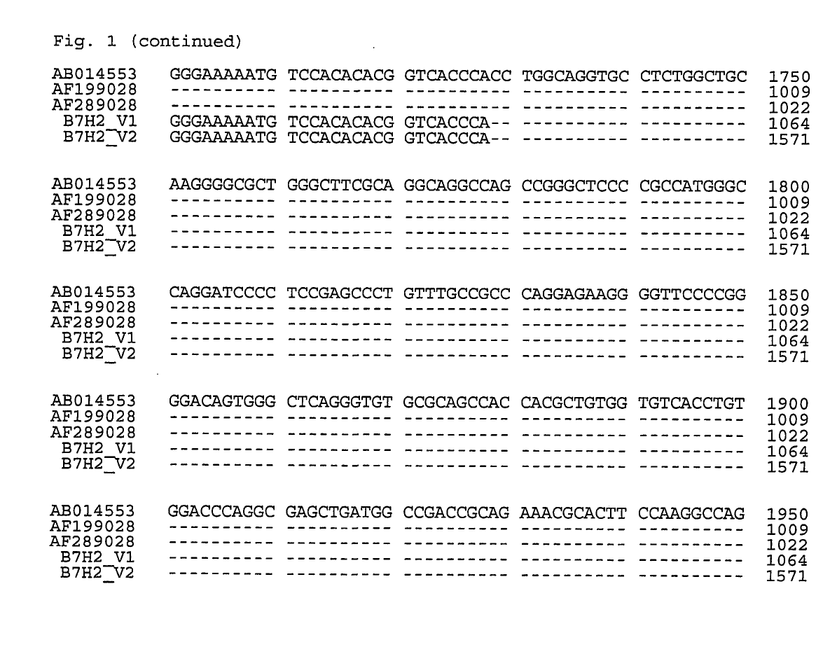

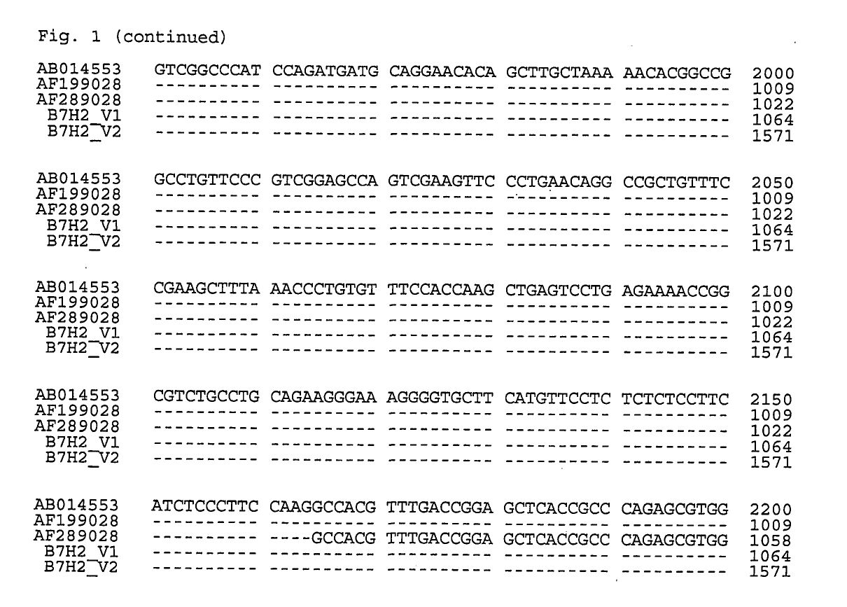

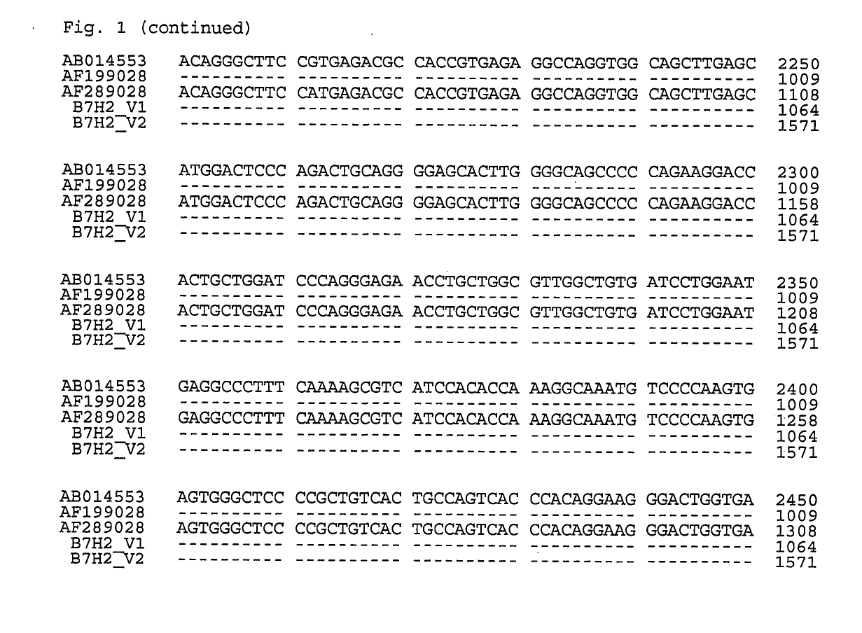

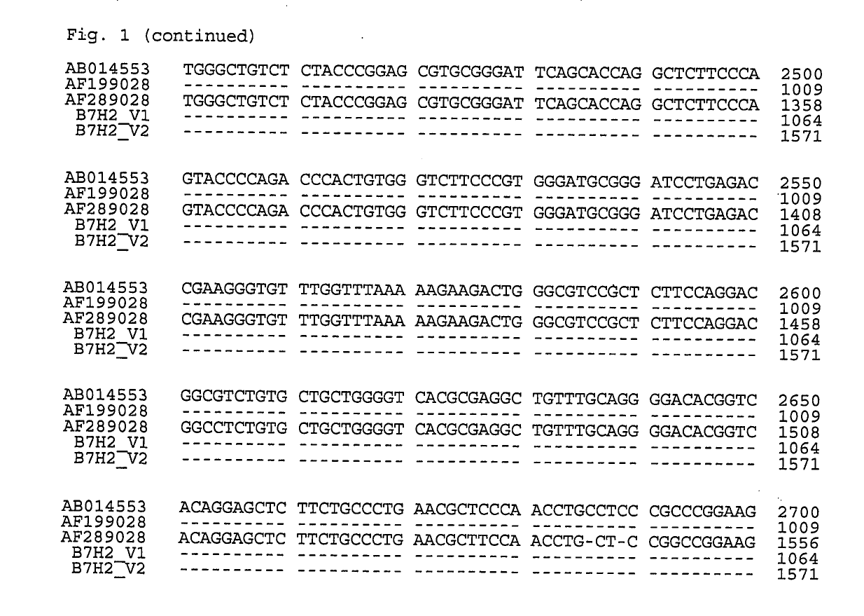

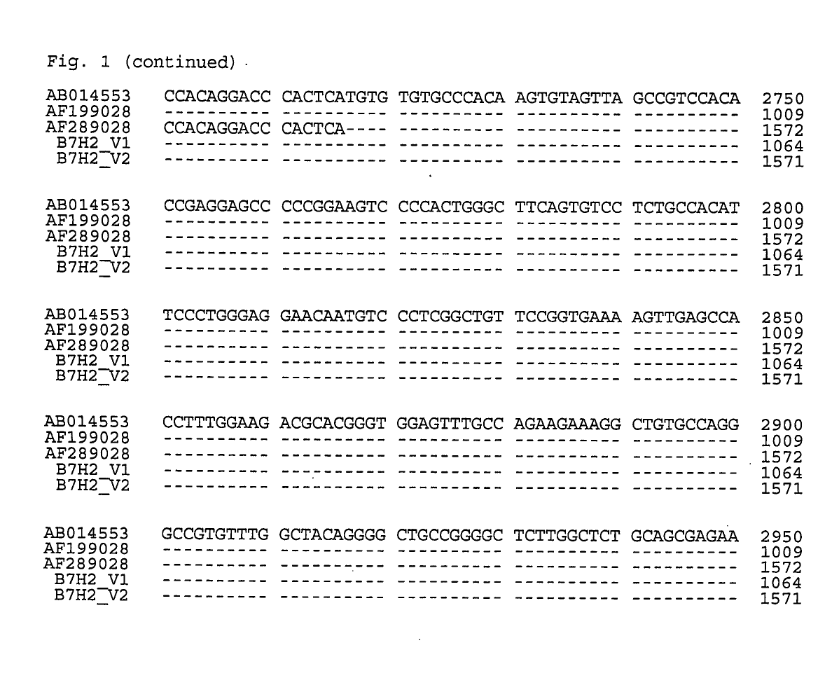

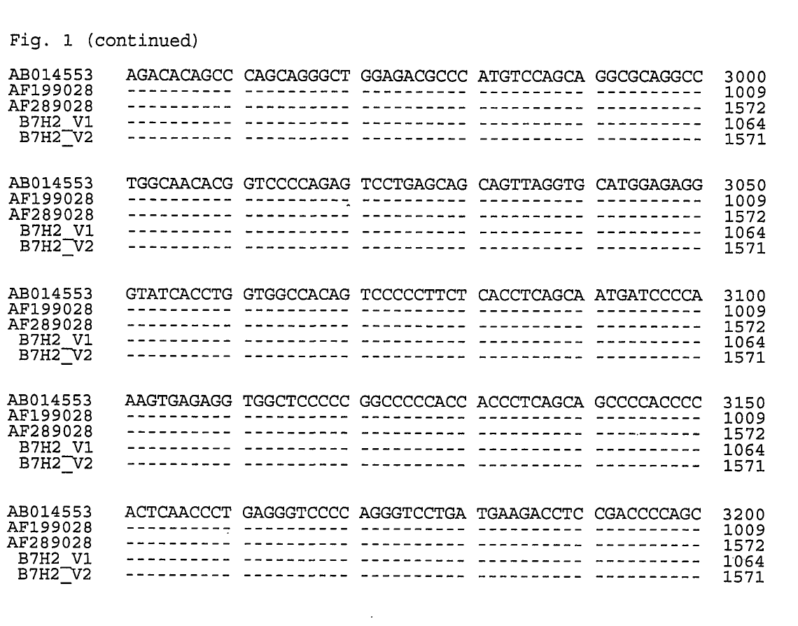

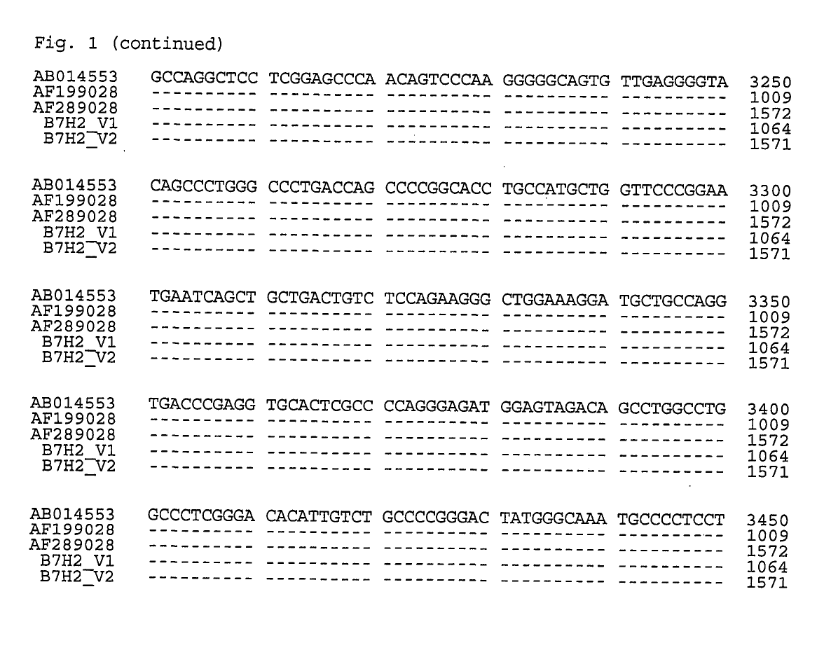

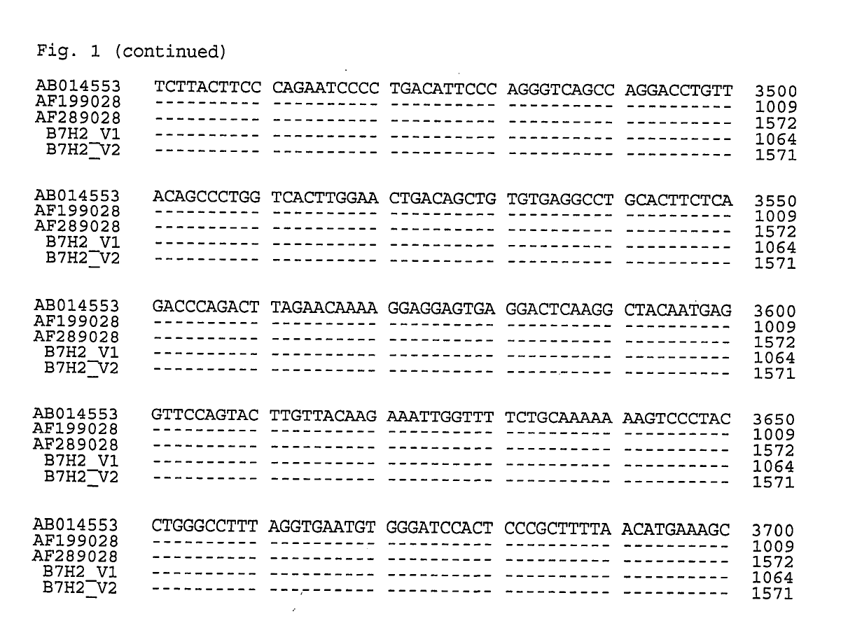

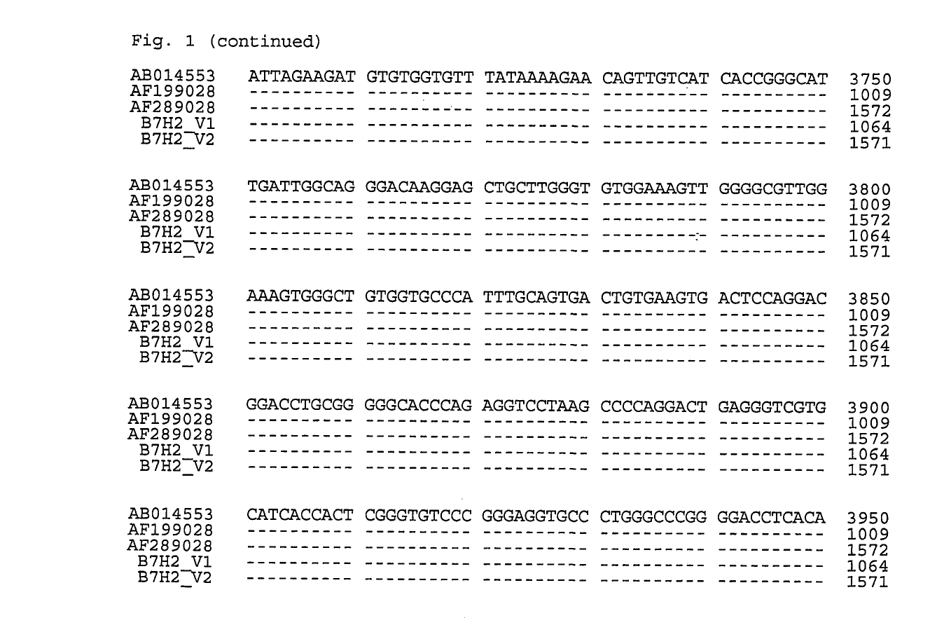

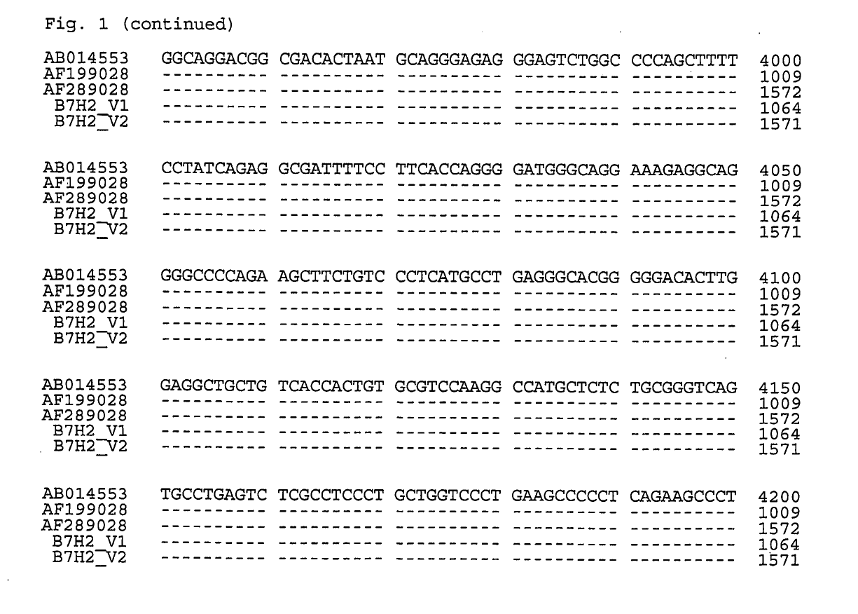

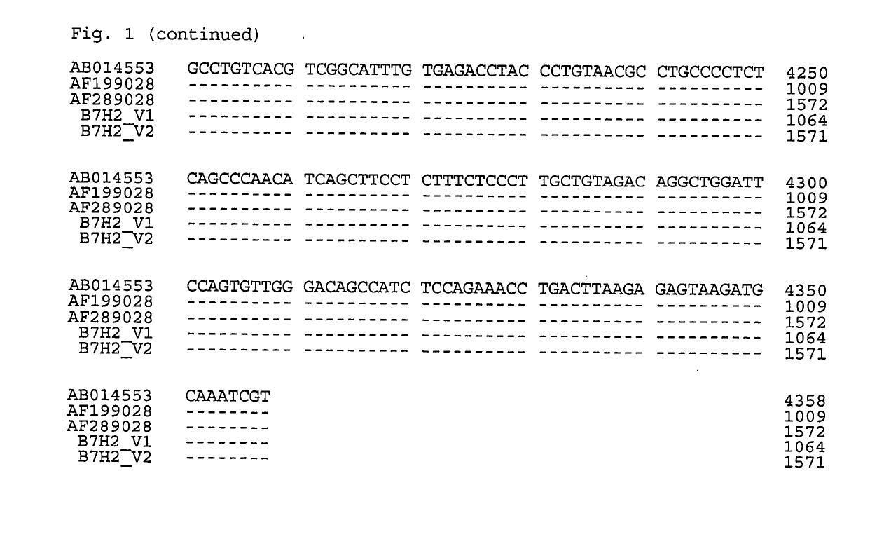

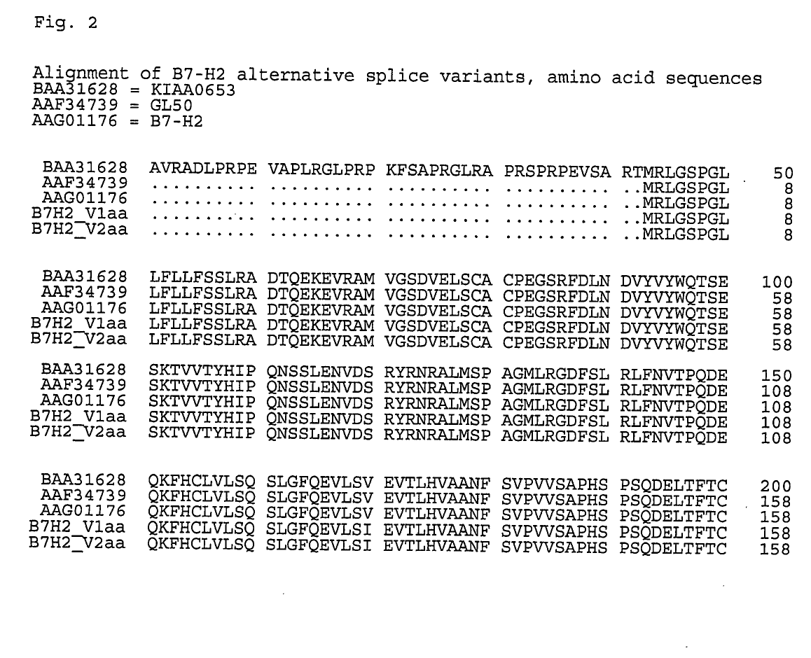

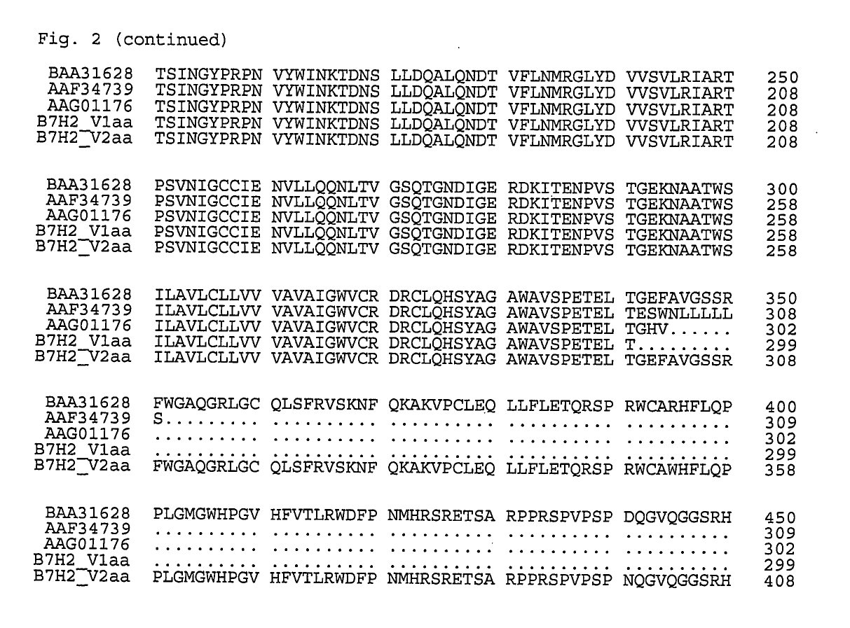

### DETAILED DESCRIPTION OF THE INVENTION

A novel human B7-H2 protein encoded by the transcript 1 (SEQ ID NO:3) (B7-H2 VI) or by the transcript 2 (SEQ ID NO:4) is a discovery of the present invention.

The sequence of transcript 1 has a coding region 912 bp in length and has a deletion of 636 bp from bases 1027 to 1662 of the KIAA0653 mRNA sequence (GenBank accession number AB014553) reported for this gene.

The sequence of transcript 2 has a coding region 1419 bp in length and has a deletion of 129 bp from bases 1452 to 1580 of the KIAA0653 sequence.

In addition, several nucleotide sequence differences outside of the deleted region are noted between transcript 1 or 2 and the original KIAA0653 sequence.

The translation of the transcript 1 clone (137-H2 VI) gives an amino acid sequence 304 residues in length. The translation of the transcript 2 clones gives an amino acid sequence 473 residues in length.

The present inventors found B7-H2 expression to be high in lymphoid tissues such as the thymus and spleen, but also noted high levels of expression in the lung and gastrointestinal tissues, suggesting that ICOS may play a role in local immune responses in mucosal tissues.

In contrast to the limited expression of ICOS, B7-H2 was found to be expressed widely in all tissues tested, with highest expression in the liver, kidney, heart, and brain. Its wide expression compared with its ICOS receptor counterpart is consistent with a role for 137-H2 in the regulation immune responses throughout the body. By being expressed in most tissues, B7-H2 generally prevent excessive deviation of immune response toward the Th1 phenotype by stimulating activated, ICOS-expressing T cells to produce cytokines that force the response back toward a more Th2 phenotype. At the same time, B7-H2 itself can transduce a signal back into the cell on which it is expressed in order to indicate to the cell that an activated T cell is close by.

The two new variants of B7-H2 differ from the published B7-H2 sequences primarily in their cytoplasmic domains. B7-H2 V1, with its 28-residue cytoplasmic tail, resembles most the amino acid sequences of GL-50 and B7-H2 with their short 33- and 26-residue cytoplasmic tails. B7-H2 V2, on the other hand, has a cytoplasmic tail of 197 residues, more similar in length to the cytoplasmic tail of K1AA0653, but lacks a 43 amino acid sequence that is repeated in tandem in K1AA0653. The differences in the cytoplasmic tails have potentially significant effects on signal transduction into the cell on which the B7-H2 molecules are expressed. The longer tail may, for example, signal into its cell to induce the production of Th2 promoting cytokines (ref. 11), and thereby amplify the ICOS-induced effects in the T cell, while the shorter tail has no such signaling function in itself but instead may interact with secondary signaling molecules or other molecules.

The differences in signaling between the various forms of B7-H2 can be expected to have important effects in immune responses to pathogens and in disease pathogenesis. In order for the body to defend itself against different pathogens, different types of immune responses are necessary. For some pathogens, such as intracellular bacteria, a predominantly Th1 type of response is required to control infection, while for others, such as helminthes or microbes present in the extracellular milieu, a Th2 type of response is required. Inappropriate polarization of immune responses can result in inadequate protection against infection, while unregulated overpolarization of responses can have harmful sequelae. In the case of intracellular bacterial infections, the counterregulatory cytokine IL-10 is secreted rapidly after infection to control Th1 responses (ref. 12). Such a response may rely on B7-H2 both to transmit signals into the cell on which it is expressed and to stimulate ICOS on T cells. Similarly, when a Th2 response is appropriate, the amplification of the response by signaling through B7-H2 and stimulation of ICOS may be necessary for adequate defense against a pathogen. On the other hand, in autoimmune diseases and allergic diseases, uncontrolled activation of the immune response causes tissue distruction, suffering, and sometimes life-threatening complications. Upregulated expression of B7-H2 following Th1 immune responses and downregulated expression of B7-H2 following Th2 immune responses is a possible method that the body can use to avoid autoimmunity and allergy after normal immune responses. The expression of different splice variants of B7-H2 may also allow different cells to respond differently according to the situation. Therefore a cell's decision on which B7-H2 variant to express and at what level may be crucial to the development and control of an appropriate immune response.

The blockage of the functions of the B7-H2 VI and B7-H2 V2 and inhibitors for it are useful in the treatment of allergic diseases, such as respiratory allergies, food allergies, asthma, and atopic dermatitis, as well as in the treatment of intracellular bacterial infections, such as tuberculosis, leprosy, listeriosis, and salmonellosis, where a downregulation of the Th2 response and a repolarization towards a Th1 response would be beneficial. The enhancement of the functions of B7-H2 V1 and B7-H2 V2 and molecules therefor are useful in the treatment of autoimmune diseases, such as multiple sclerosis, rheumatoid arthritis, and type I diabetes, as well as in the treatment of helminth and extracellular microbial infections, where a repolarization towards a Th2 response is beneficial.

Polypeptides

Human B7-H2 polypeptides according to the invention comprise at least 6, 10, 15, 20, 25, 50, 75, 100, 125, 150, 175, 200, 225, 250, 275, 300 or 304 contiguous amino acids selected from the amino acid sequence shown in SEQ ID NO:3 or a biologically active variant thereof, as defined below. Alternatively, the human B7-H2 polypeptides of the present invention comprise at least 6, 10, 15, 20, 25, 50, 75, 100, 125, 150, 175, 200, 225, 250, 275, 300, 325, 350, 375, 400, 425, 450, or 473 contiguous amino acids selected from the amino acid sequence shown in SEQ ID NO:4 or biologically active variant thereof as defined below. A human B7-H2 polypeptide of the invention therefore can be a portion of a human B7-H2, a full-length human B7-H2, or a fusion protein comprising all or a portion of a human B7-H2.

Biologically Active Variants ofB7-H2 V

Human B7-H2 V polypeptide variants which are biologically active, e.g., retain a ICOS binding activity, also are human B7-H2 V polypeptides. Preferably, naturally or non-naturally occurring human B7-H2 polypeptide variants have amino acid sequences which are at least about 31, 35, 40, 45, 50, 55, 60, 65, or 70, preferably about 75, 80, 85, 90, 96, 96, or 98% identical to the amino acid sequence shown in SEQ ID NO:3, or SEQ ID NO:4 or a fragment thereof. Percent identity between a putative human B7-H2 polypeptide variant and an amino acid sequence of SEQ ID NO:3 or 4 is determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (ibid.). Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative variant. The FASTA algorithm is described y Pearson and Lipman, Proc. Nat'l Acad. Sci. USA 85:2444(1988), and by Pearson, Meth. Enzymol. 183:63 (1990). Briefly, FASTA first characterizes sequence similarity by identifying regions shared by the query sequence (e.g. SEQ ID NO: 2) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “tmmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to for man approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Preferred parameters for FASTA analysis are: ktup=1, gapopeningpenalty=10, gap extension penalty=1, and substitution matrix-BLOSUM62. These parameters can be introduced into a FASTA program by modifying the scoring matrix file (“SMATRIX”), as explained in Appendix 2 of Pearson, Meth. Enzymol. 183:63 (1990). FASTA can also be used to determine the sequence identity of nucleic acid molecules using a ratio as disclosed above. For nucleotide sequence comparisons, the ktup value can range between one to six, preferably from three to six, most preferably three, with other parameters set as default.

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of a human B7-H2 polypeptide can be found using computer programs well known in the art, such as DNASTAR software. Whether an amino acid change results in a biologically active human B7-H2 polypeptide can readily be determined by assaying for Shh-binding activity, as described for example, in Carpenter, et al., PROC. NATL. ACAD. SCI. USA. 95, 13630-34 (1998).

Fusion Proteins

Fusion proteins are useful for generating antibodies against human B7-H2 polypeptide amino acid sequences and for use in various assay systems. For example, fusion proteins can be used to identify proteins that interact with portions of a human B7-H2 polypeptide. Protein affinity chromatography or library-based assays for protein-protein interactions, such as the yeast two-hybrid or phage display systems, can be used for this purpose. Such methods are well known in the art and also can be used as drug screens.

A human B7-H2 polypeptide fusion protein comprises two polypeptide segments fused together by means of a peptide bond. The first polypeptide segment comprises at least 6, 10, 15, 20, 25, 50, 75, 100, 125, 150, 175, 200, 225, 250, 275, 300 or 304 contiguous amino acids of SEQ IID NO:3 or of a biologically active variant, such as those described above. Alternatively, the first polypeptide segment comprises at least 6, 10, 15, 20, 25, 50, 75, 100, 125, 150, 175, 200, 225, 250, 275, 300, 325, 350, 375, 400, 425, 450, or 473 contiguous amino acids of SEQ ID NO:4 or of a biologically active variant, such as those described above. The first polypeptide segment also can comprise full-length human B7-H2 V2.

The second polypeptide segment can be a full-length protein or a protein fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags are used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions. A fusion protein also can be engineered to contain a cleavage site located between the human B7-H2 polypeptide-encoding sequence and the heterologous protein sequence, so that the human B7-H2 polypeptide can be cleaved and purified away from the heterologous moiety.

A fusion protein can be synthesized chemically, as is known in the art. Preferably, a fusion protein is produced by covalently linking two polypeptide segments or by standard procedures in the art of molecular biology. Recombinant DNA methods can be used to prepare fusion proteins, for example, by making a DNA construct which comprises coding sequences selected from the complement of SEQ ID NO:1 or 2 in proper reading frame with nucleotides encoding the second polypeptide segment and expressing the DNA construct in a host cell, as is known in the art. Many kits for constructing fusion proteins are available from companies such as Promega Corporation (Madison, Wis.), Stratagene (La Jolla, Calif.), CLONTECH (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Identification of Species Homologues

Species homologues of human B7-H2 polypeptide can be obtained using human B7-H2 polypeptide polynucleotides (described below) to make suitable probes or primers for screening cDNA expression libraries from other species, such as mice, monkeys, or yeast, identifying cDNAs which encode homologues of human B7-H2 polypeptide, and expressing the cDNAs as is known in the art.

Polynucleotides

A human B7-H2 polynucleotide can be single- or double-stranded and comprises a coding sequence or the complement of a coding sequence for a human B7-H2 polypeptide. A coding sequence for human B7-H2 is shown in SEQ ID NO:1 or 2.

Degenerate nucleotide sequences encoding human B7-H2 polypeptides, as well as homologous nucleotide sequences which are at least about 50, 55, 60, 65, 70, preferably about 75, 90, 96, or 98% identical to the nucleotide sequence shown in SEQ ID NO:1 or 2 or their complement also are human B7-H2 polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologues, and variants of human B7-H2 polynucleotides that encode biologically active human B7-H2 polypeptides also are human B7-H2 polynucleotides. Fragments comprising 8, 10, 12, 15, 20, or 25 contiguous nucleotides of SEQ ID NO:1 or 2 or their complement also are human B7-H2 polynucleotides. Such polynucleotides can be used, for example, as antisense oligonucleotides or as hybridization probes.

Identification of Polynucleotide Variants and Homologues

Variants and homologues of the human B7-H2 polynucleotides described above also are human B7-H2 polynucleotides. Typically, homologous human B7-H2 polynucleotide sequences can be identified by hybridization of candidate polynucleotides to known human B7-H2 polynucleotides under stringent conditions, as is known in the art. For example, using the following wash conditions—2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each—homologous sequences can be identified which contain at most about 25-30% basepair mismatches. More preferably, homologous nucleic acid strands contain 15-25% basepair mismatches, even more preferably 5-15% basepair mismatches.

Species homologues of the human B7-H2 polynucleotides disclosed herein also can be identified by making suitable probes or primers and screening cDNA expression libraries from other species, such as mice, monkeys, or yeast. Human variants of human B7-H2 polynucleotides can be identified, for example, by screening human cDNA expression libraries. It is well known that the Tₘ of a double-stranded DNA decreases by 1-1.5° C. with every 1% decrease in homology (Bonner et al., J. Mol. Biol. 81, 123 (1973). Variants of human B7-H2 polynucleotides or human B7-H2 polynucleotides of other species can therefore be identified by hybridizing a putative homologous human B7-H2 polynucleotide with a polynucleotide having a nucleotide sequence of SEQ ID NO:1 or 2, or the complement thereof to form a test hybrid. The melting temperature of the test hybrid is compared with the melting temperature of a hybrid comprising polynucleotides having perfectly complementary nucleotide sequences, and the number or percent of basepair mismatches within the test hybrid is calculated.

Nucleotide sequences which hybridize to human B7-H2 polynucleotides or their complements following stringent hybridization and/or wash conditions also are human B7-H2 polynucleotides. Stringent wash conditions are well known and understood in the art and are disclosed, for example, in Sambrook et al., MOLECULAR CLONING: A LABORATORY MANUAL, 2d ed., 1989, at pages 9.50-9.51.

Typically, for stringent hybridization conditions a combination of temperature and salt concentration should be chosen that is approximately 12-20° C. below the calculated Tₘ of the hybrid under study. The Tₘ of a hybrid between a human B7-H2 polynucleotide having a nucleotide sequence shown in SEQ ID NO:1 or 2, or the complement thereof and a polynucleotide sequence which is at least about 50, preferably about 75, 90, 96, or 98% identical to one of those nucleotide sequences can be calculated, for example, using the equation of Bolton and McCarthy, Proc. Natl. Acad. Sci. U.S.A. 48, 1390 (1962):

Tₘ=81.5° C.−16.6(log₁₀[Na⁺])+0.41(% G+C)−0.63(% formamide)−600/l), where l=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C.

Preparation of Polynucleotides

A human B7-H2 polynucleotide can be isolated free of other cellular components such as membrane components, proteins, and lipids. Polynucleotides can be made by a cell and isolated using standard nucleic acid purification techniques, or synthesized using an amplification technique, such as the polymerase chain reaction (PCR), or by using an automatic synthesizer. Methods for isolating polynucleotides are routine and are known in the art. Any such technique for obtaining a polynucleotide can be used to obtain isolated human B7-H2 polynucleotides. For example, restriction enzymes and probes can be used to isolate polynucleotide fragments which comprises B7-H2 like nucleotide sequences. Isolated polynucleotides are in preparations which are free or at least 70, 80, or 90% free of other molecules.

Human B7-H2 cDNA molecules can be made with standard molecular biology techniques, using human B7-H2 mRNA as a template. Human B7-H2 cDNA molecules can thereafter be replicated using molecular biology techniques known in the art and disclosed in manuals such as Sambrook et al. (1989). An amplification technique, such as PCR, can be used to obtain additional copies of polynucleotides of the invention, using either human genomic DNA or cDNA as a template.

Alternatively, synthetic chemistry techniques can be used to synthesizes human B7-H2 polynucleotides. The degeneracy of the genetic code allows alternate nucleotide sequences to be synthesized which will encode a human B7-H2 polypeptide having, for example, an amino acid sequence shown in SEQ ID NO:1 or 2 or a biologically active variant thereof.

Extending Polynucleotides

Various PCR-based methods can be used to extend the nucleic acid sequences disclosed herein to detect upstream sequences such as promoters and regulatory elements. For example, restriction-site PCR uses universal primers to retrieve unknown sequence adjacent to a known locus (Sarkar, PCR Methods Applic. 2, 318-322, 1993). Genomic DNA is first amplified in the presence of a primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR also can be used to amplify or extend sequences using divergent primers based on a known region (Triglia et al., Nucleic Acids Res. 16, 8186, 1988). Primers can be designed using commercially available software, such as OLIGO 4.06 Primer Analysis software (National Biosciences Inc., Plymouth, Minn.), to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template.

Another method which can be used is capture PCR, which involves PCR amplification of DNA fragments adjacent to a known sequence in human and yeast artificial chromosome DNA (Lagerstrom et al., PCR Methods Applic. 1, 111-119, 1991). In this method, multiple restriction enzyme digestions and ligations also can be used to place an engineered double-stranded sequence into an unknown fragment of the DNA molecule before performing PCR.

Another method which can be used to retrieve unknown sequences is that of Parker et al., Nucleic Acids Res. 19, 3055-3060, 1991). Additionally, PCR, nested primers, and PROMOTERFINDER libraries (CLONTECH, Palo Alto, Calif) can be used to walk genomic DNA (CLONTECH, Palo Alto, Calif). This process avoids the need to screen libraries and is useful in finding intron/exon junctions.

When screening for full-length cDNAs, it is preferable to use libraries that have been size-selected to include larger cDNAs. Randomly-primed libraries are preferable, in that they will contain more sequences which contain the 5′ regions of genes. Use of a randomly primed library may be especially preferable for situations in which an oligo d(T) library does not yield a full-length cDNA-Genomic libraries can be useful for extension of sequence into 5′ non-transcribed regulatory regions.

Commercially available capillary electrophoresis systems can be used to analyze the size or confirm the nucleotide sequence of PCR or sequencing products. For example, capillary sequencing can employ flowable polymers for electrophoretic separation, four different fluorescent dyes (one for each nucleotide) which are laser activated, and detection of the emitted wavelengths by a charge coupled device camera. Output/light intensity can be converted to electrical signal using appropriate software (e.g. GENOTYPER and Sequence NAVIGATOR, Perkin Elmer), and the entire process from loading of samples to computer analysis and electronic data display can be computer controlled. Capillary electrophoresis is especially preferable for the sequencing of small pieces of DNA that might be present in limited amounts in a particular sample.

Obtaining Polypeptides

Human B7-H2 polypeptides can be obtained, for example, by purification from human cells, by expression of human B7-H2 polynucleotides, or by direct chemical synthesis.

Protein Purification

Human B7-H2 polypeptides can be purified from any cell which expresses the molecule, including host cells which have been transfected with human B7-H2 expression constructs. A purified human B7-H2 polypeptide is separated from other compounds which normally associate with the human B7-H2 polypeptide in the cell, such as certain proteins, carbohydrates, or lipids, using methods well-known in the art. Such methods include, but are not limited to, size exclusion chromatography, ammonium sulfate fractionation, ion exchange chromatography, affinity chromatography, and preparative gel electrophoresis. A preparation of purified human B7-H2 polypeptides is at least 80% pure; preferably, the preparations are 90%, 95%, or 99% pure. Purity of the preparations can be assessed by any means known in the art, such as SDS-polyacrylamide gel electrophoresis.

Expression of Polynucleotides

To express a human B7-H2 polynucleotide, the polynucleotide can be inserted into an expression vector that contains the necessary elements for the transcription and translation of the inserted coding sequence. Methods that are well known to those skilled in the art can be used to construct expression vectors containing sequences encoding human B7-H2 polypeptides and appropriate transcriptional and translational control elements. These methods include in vitro recombinant DNA techniques, synthetic techniques, and in vivo genetic recombination. Such techniques are described, for example, in Sambrook et al. (1989) and in Ausubel et al., CURRENT PROTOCOLS IN MOLECULAR BIOLOGY, John Wiley & Sons, New York, N.Y., 1989.

A variety of expression vector/host systems can be utilized to contain and express sequences encoding a human B7-H2 polypeptide. These include, but are not limited to, microorganisms, such as bacteria transformed with recombinant bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors (e.g., Ti or pBR322 plasmids), or animal cell systems.

The control elements or regulatory sequences are those non-translated regions of the vector—enhancers, promoters, 5′ and 3′ untranslated regions—which interact with host cellular proteins to carry out transcription and translation. Such elements can vary in their strength and specificity. Depending on the vector system and host utilized, any number of suitable transcription and translation elements, including constitutive and inducible promoters, can be used. For example, when cloning in bacterial systems, inducible promoters such as the hybrid lacZ promoter of the BLUESCRIPT phagemid (Stratagene, LaJolla, Calif.) or pSPORT1 plasmid (Life Technologies) and the like can be used. The baculovirus polyhedrin promoter can be used in insect cells. Promoters or enhancers derived from the genomes of plant cells (e.g., heat shock, RUBISCO, and storage protein genes) or from plant viruses (e.g., viral promoters or leader sequences) can be cloned into the vector. In mammalian cell systems, promoters from mammalian genes or from mammalian viruses are preferable. If it is necessary to generate a cell line that contains multiple copies of a nucleotide sequence encoding a human B7-H2 polypeptide, vectors based on SV40 or EBV can be used with an appropriate selectable marker.

Bacterial and Yeast Expression Systems

In bacterial systems, a number of expression vectors can be selected depending upon the use intended for the human B7-H2 polypeptide. For example, when a large quantity of a human B7-H2 polypeptide is needed for the induction of antibodies, vectors which direct high level expression of fusion proteins that are readily purified can be used. Such vectors include, but are not limited to, multifunctional E. coli cloning and expression vectors such as BLUESCRIPT (Stratagene). In a BLUESCRIPT vector, a sequence encoding the human B7-H2 polypeptide can be ligated into the vector in frame with sequences for the amino-terminal Met and the subsequent 7 residues of β-galactosidase so that a hybrid protein is produced. pIN vectors (Van Heeke & Schuster, J. Biol. Chem. 264, 5503-5509, 1989) or pGEX vectors (Promega, Madison, Wis.) also can be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems can be designed to include heparin, thrombin, or factor Xa protease cleavage sites so that the cloned polypeptide of interest can be released from the GST moiety at will.

In the yeast Saccharoniyces cerevisiae, a number of vectors containing constitutive or inducible promoters such as alpha factor, alcohol oxidase, and PGH can be used. For reviews, see Ausubel et al. (1989) and Grant et al., Methods Enzymol. 153, 516-544, 1987.

Plant and Insect Expression Systems

If plant expression vectors are used, the expression of sequences encoding human B7-H2 polypeptides can be driven by any of a number of promoters. For example, viral promoters such as the 35S and 19S promoters of CaMV can be used alone or in combination with the omega leader sequence from TMV (Takamatsu, EMBO J. 6, 307-311, 1987). Alternatively, plant promoters such as the small subunit of RUBISCO or heat shock promoters can be used (Coruzzi et al., EMBO J. 3, 1671-1680, 1984; Broglie et al., Science 224, 838-843, 1984; Winter et al., Results Probl. Cell Differ. 17, 85-105, 1991). These constructs can be introduced into plant cells by direct DNA transformation or by pathogen-mediated transfection. Such techniques are described in a number of generally available reviews (e.g., Hobbs or Murray, in MCGRAW HILL YEARBOOK OF SCIENCE AND TECHNOLOGY, McGraw Hill, New York, N.Y., pp. 191-196, 1992).

An insect system also can be used to express a human B7-H2 polypeptide. For example, in one such system Autographa californica nuclear polyhedrosis virus (AcNPV) is used as a vector to express foreign genes in Spodoptera frugiperda cells or in Trichoplusia larvae. Sequences encoding human B7-H2 polypeptides can be cloned into a non-essential region of the virus, such as the polyhedrin gene, and placed under control of the polyhedrin promoter. Successful insertion of human B7-H2 polypeptides will render the polyhedrin gene inactive and produce recombinant virus lacking coat protein. The recombinant viruses can then be used to infect S. frugiperda cells or Trichoplusia larvae in which human B7-H2 polypeptides can be expressed (Engelhard et al., Proc. Nat. Acad. Sci. 91, 3224-3227, 1994).

### Mammalian Expression Systems

A number of viral-based expression systems can be used to express human B7-H2 polypeptides in mammalian host cells. For example, if an adenovirus is used as an expression vector, sequences encoding human B7-H2 polypeptides can be ligated into an adenovirus transcription/translation complex comprising the late promoter and tripartite leader sequence. Insertion in a non-essential E1 or E3 region of the viral genome can be used to obtain a viable virus which is capable of expressing a human B7-H2 polypeptide in infected host cells (Logan & Shenk, Proc. Natl. Acad. Sci. 81, 3655-3659, 1984). If desired, transcription enhancers, such as the Rous sarcoma virus (RSV) enhancer, can be used to increase expression in mammalian host cells.

Human artificial chromosomes (HACs) also can be used to deliver larger fragments of DNA than can be contained and expressed in a plasmid. HACs of 6M to 10M are constructed and delivered to cells via conventional delivery methods (e.g., liposomes, polycationic amino polymers, or vesicles).

Specific initiation signals also can be used to achieve more efficient translation of sequences encoding human B7-H2 polypeptides. Such signals include the ATG initiation codon and adjacent sequences. In cases where sequences encoding a human B7-H2 polypeptide, its initiation codon, and upstream sequences are inserted into the appropriate expression vector, no additional transcriptional or translational control signals may be needed. However, in cases where only coding sequence, or a fragment thereof is inserted, exogenous translational control signals (including the ATG initiation codon) should be provided. The initiation codon should be in the correct reading frame to ensure translation of the entire insert. Exogenous translational elements and initiation codons can be of various origins, both natural and synthetic. The efficiency of expression can be enhanced by the inclusion of enhancers which are appropriate for the particular cell system which is used (see Scharf et al., Results Probl. Cell Differ. 20, 125-162, 1994).

Host Cells

A host cell strain can be chosen for its ability to modulate the expression of the inserted sequences or to process the expressed human B7-H2 polypeptide in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, and WI38), are available from the American Type Culture Collection (ATCC; 10801 University Boulevard, Manassas, Va. 20110-2209) and can be chosen to ensure the correct modification and processing of the foreign protein.

Stable expression is preferred for long-term, high-yield production of recombinant proteins. For example, cell lines which stably express human B7-H2 polypeptides can be transformed using expression vectors which can contain viral origins of replication and/or endogenous expression elements and a selectable marker gene on the same or on a separate vector. Following the introduction of the vector, cells can be allowed to grow for 1-2 days in an enriched medium before they are switched to a selective medium. The purpose of the selectable marker is to confer resistance to selection, and its presence allows growth and recovery of cells which successfully express the introduced human B7-H2 sequences. Resistant clones of stably transformed cells can be proliferated using tissue culture techniques appropriate to the cell type. See, for example, ANIMAL CELL CULTURE, R. I. Freshney, ed., 1986.

Any number of selection systems can be used to recover transformed cell lines. These include, but are not limited to, the herpes simplex virus thymidine kinase (Wigler et al., Cell 11, 223-32, 1977) and adenine phosphoribosyltransferase (Lowy et al., Cell 22, 817-23, 1980) genes which can be employed in tk⁻ or aprf⁻ cells, respectively. Also, antimetabolite, antibiotic, or herbicide resistance can be used as the basis for selection. For example, dhfr confers resistance to methotrexate (Wigler et al., Proc. Natl. Acad. Sci. 77, 3567-70, 1980), npt confers resistance to the aminoglycosides, neomycin and G-418 (Colbere-Garapin et al., J. Mol. Biol. 150, 1-14, 1981), and als and pat confer resistance to chlorsulfuron and phosphinotricin acetyltransferase, respectively (Murray, 1992, supra). Additional selectable genes have been described. For example, trpB allows cells to utilize indole in place of tryptophan, or hisD, which allows cells to utilize histinol in place of histidine (Hartman & Mulligan, Proc. Natl. Acad. Sci. 85, 8047-51, 1988). Visible markers such as anthocyanins, β-glucuronidase and its substrate GUS, and luciferase and its substrate luciferin, can be used to identify transformants and to quantify the amount of transient or stable protein expression attributable to a specific vector system (Rhodes et al., Methods Mol. Biol. 55, 121-131, 1995).

Detecting Expression

Although the presence of marker gene expression suggests that the human B7-H2 polynucleotide is also present, its presence and expression may need to be confirmed. For example, if a sequence encoding a human B7-H2 polypeptide is inserted within a marker gene sequence, transformed cells containing sequences which encode a human B7-H2 polypeptide can be identified by the absence of marker gene function. Alternatively, a marker gene can be placed in tandem with a sequence encoding a human B7-H2 polypeptide under the control of a single promoter. Expression of the marker gene in response to induction or selection usually indicates expression of the human B7-H2 polynucleotide.

Alternatively, host cells which contain a human B7-H2 polynucleotide and which express a human B7-H2 polypeptide can be identified by a variety of procedures known to those of skill in the art. These procedures include, but are not limited to, DNA-DNA or DNA-RNA hybridizations and protein bioassay or immunoassay techniques which include membrane, solution, or chip-based technologies for the detection and/or quantification of nucleic acid or protein. For example, the presence of a polynucleotide sequence encoding a human B7-H2 polypeptide can be detected by DNA-DNA or DNA-RNA hybridization or amplification using probes or fragments or fragments of polynucleotides encoding a human B7-H2 polypeptide. Nucleic acid amplification-based assays involve the use of oligonucleotides selected from sequences encoding a human B7-H2 polypeptide to detect transformants which contain a human B7-H2 polynucleotide.

A variety of protocols for detecting and measuring the expression of a human B7-H2 polypeptide, using either polyclonal or monoclonal antibodies specific for the polypeptide, are known in the art. Examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on a human B7-H2 polypeptide can be used, or a competitive binding assay can be employed. These and other assays are described in Hampton et al., SEROLOGICAL METHODS: A LABORATORY MANUAL, APS Press, St. Paul, Minn., 1990) and Maddox et al., J. Exp. Med 158, 1211-1216, 1983).

A wide variety of labels and conjugation techniques are known by those skilled in the art and can be used in various nucleic acid and amino acid assays. Means for producing labeled hybridization or PCR probes for detecting sequences related to polynucleotides encoding human B7-H2 polypeptides include oligolabeling, nick translation, end-labeling, or PCR amplification using a labeled nucleotide. Alternatively, sequences encoding a human B7-H2 polypeptide can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, magnetic particles, and the like.

Expression and Purification of Polypeptides

Host cells transformed with nucleotide sequences encoding a human B7-H2 polypeptide can be cultured under conditions suitable for the expression and recovery of the protein from cell culture. The polypeptide produced by a transformed cell can be secreted or contained intracellularly depending on the sequence and/or the vector used. As will be understood by those of skill in the art, expression Vectors containing polynucleotides which encode human B7-H2 polypeptides can be designed to contain signal sequences which direct secretion of soluble human B7-H2 polypeptides through a prokaryotic or eukaryotic cell membrane or which direct the membrane insertion of membrane-bound human B7-H2 polypeptide.

As discussed above, other constructions can be used to join a sequence encoding a human B7-H2 polypeptide to a nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). Inclusion of cleavable linker sequences such as those specific for Factor Xa or enterokinase (Invitrogen, San Diego, Calif.) between the purification domain and the human B7-H2 polypeptide also can be used to facilitate purification. One such expression vector provides for expression of a fusion protein containing a human B7-H2 polypeptide and 6 histidine residues preceding a thioredoxin or an enterokinase cleavage site. The histidine residues facilitate purification by IMAC (immobilized metal ion affinity chromatography, as described in Porath et al., Prot. Exp. Purif 3, 263-281, 1992), while the enterolinase cleavage site provides a means for purifying the _human B7-H2 polypeptide from the fusion protein. Vectors which contain fusion proteins are disclosed in Kroll et al., DNA Cell Biol. 12, 441-453, 1993.

Chemical Synthesis

Sequences encoding a human B7-H2 polypeptide can be synthesized, in whole or in part, using chemical methods well known in the art (see Caruthers et al., Nucl. Acids Res. Symp. Ser. 215-223, 1980; Horn et al. Nucl. Acids Res. Symp. Ser. 225-232, 1980). Alternatively, a human B7-H2 polypeptide itself can be produced using chemical methods to synthesize its amino acid sequence, such as by direct peptide synthesis using solid-phase techniques (Merrifield, J. Am. Chem. Soc. 85, 2149-2154, 1963; Roberge et al., Science 269, 202-204, 1995). Protein synthesis can be performed using manual techniques or by automation. Automated synthesis can be achieved, for example, using Applied Biosystems 431A Peptide Synthesizer (Perlin Elmer). Optionally, fragments of human B7-H2 polypeptides can be separately synthesized and combined using chemical methods to produce a full-length molecule.

The newly synthesized peptide can be substantially purified by preparative high performance liquid chromatography (e.g., Creighton, PROTEINS: STRUCTURES AND MOLECULAR PRINCIPLES, WH Freeman and Co., New York, N.Y., 1983). The composition of a synthetic human B7-H2 polypeptide can be confirmed by amino acid analysis or sequencing (e.g., the Edman degradation procedure; see Creighton, supra). Additionally, any portion of the amino acid sequence of the human B7-H2 polypeptide can be altered during direct synthesis and/or combined using chemical methods with sequences from other proteins to produce a variant polypeptide or a fusion protein.

Production of Altered Polypeptides

As will be understood by those of skill in the art, it may be advantageous to produce human B7-H2 polypeptide-encoding nucleotide sequences possessing non-naturally occurring codons. For example, codons preferred by a particular prokaryotic or eukaryotic host can be selected to increase the rate of protein expression or to produce an RNA transcript having desirable properties, such as a half-life which is longer than that of a transcript generated from the naturally occurring sequence.

The nucleotide sequences disclosed herein can be engineered using methods generally known in the art to alter human B7-H2 polypeptide-encoding sequences for a variety of reasons, including but not limited to, alterations which modify the cloning, processing, and/or expression of the polypeptide or mRNA product. DNA shuffling by random fragmentation and PCR reassembly of gene fragments and synthetic oligonucleotides can be used to engineer the nucleotide sequences. For example, site-directed mutagenesis can be used to insert new restriction sites, alter glycosylation patterns, change codon preference, produce splice variants, introduce mutations, and so forth.

Antibodies

Any type of antibody known in the art can be generated to bind specifically to an epitope of a human B7-H2 polypeptide. “Antibody” as used herein includes intact immunoglobulin molecules, as well as fragments thereof, such as Fab, F(ab′)₂, and Fv, which are capable of binding an epitope of a human B7-H2 polypeptide. Typically, at least 6, 8, 10, or 12 contiguous amino acids are required to form an epitope. However, epitopes which involve non-contiguous amino acids may require more, e.g., at least 15, 25, or 50 amino acids. An antibody which specifically binds to an epitope of a human B7-H2 polypeptide can be used therapeutically, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen.

Typically, an antibody which specifically binds to a human B7-H2 polypeptide provides a detection signal at least 5-, 10-, or 20-fold higher than a detection signal provided with other proteins when used in an immunochemical assay. Preferably, antibodies which specifically bind to human B7-H2 like polypeptides do not detect other proteins in immunochemical assays and can immunoprecipitate a human B7-H2 polypeptide from solution.

Human B7-H2 polypeptides can be used to immunize a mammal, such as a mouse, rat, rabbit, guinea pig, monkey, or human, to produce polyclonal antibodies. If desired, a human B7-H2 polypeptide can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a human B7-H2 polypeptide can be prepared using any technique which provides for the production of antibody molecules by continuous cell lines in culture. These techniques include, but are not limited to, the hybridoma technique, the human B-cell hybridoma technique, and the EBV-hybridoma technique (Kohler et al., Nature 256, 495-497, 1985; Kozbor et al., J. Immunol. Methods 81, 3142, 1985; Cote et al., Proc. Natl. Acad. Sci. 80, 2026-2030, 1983; Cole et al., Mol. Cell Biol. 62, 109-120, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad Sci. 81, 6851-6855, 1984; Neuberger et al., Nature 312, 604-608, 1984; Takeda et al., Nature 314, 452-454, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions. Alternatively, humanized antibodies can be produced using recombinant methods, as described in GB2188638B. Antibodies which specifically bind to a human B7-H2 polypeptide can contain antigen binding sites which are either partially or fully humanized, as disclosed in U.S. Pat. No. 5,565,332.

Alternatively, techniques described for the production of single chain antibodies can be adapted using methods known in the art to produce single chain antibodies which specifically bind to human B7-H2 polypeptides. Antibodies with related specificity, but of distinct idiotypic composition, can be generated by chain shuffling from random combinatorial immunoglobin libraries (Burton, Proc. Natl. Acad. Sci. 88, 11120-23, 1991).

Single-chain antibodies also can be constructed using a DNA amplification method, such as PCR, using hybridoma cDNA as a template (Thirion et al., 1996, Eur. J. Cancer Prev. 5, 507-11). Single-chain antibodies can be mono- or bispecific, and can be bivalent or tetravalent. Construction of tetravalent, bispecific single-chain antibodies is taught, for example, in Coloma & Morrison, 1997, Nat. Biotechnol. 15, 159-63. Construction of bivalent, bispecific single-chain antibodies is taught in Mallender & Voss, 1994, J. Biol. Chem. 269, 199-206.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., 1995, Int. J. Cancer 61, 497-501; Nicholls et al., 1993, J. Immunol. Meth. 165, 81-91).

Antibodies which specifically bind to human B7-H2 polypeptides also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833-3837, 1989; Winter et al., Nature 349, 293-299, 1991).

Other types of antibodies can be constructed and used therapeutically in methods of the invention. For example, chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies according to the invention can be purified by methods well known in the art. For example, antibodies can be affinity purified by passage over a column to which a human B7-H2 polypeptide is bound. The bound antibodies can then be eluted from the column using a buffer with a high salt concentration.

Antisense Oligonucleotides

Antisense oligonucleotides are nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of human B7-H2 gene products in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbarnates, acetamidate, carboxymethyl esters, carbonates, and phosphate triesters. See Brown, Meth Mol. Biol. 20, 1-8, 1994; Sonveaux, Meth Mol. Biol. 26, 1-72, 1994; Uhlmann et al., Chem. Rev. 90, 543-583, 1990.

Modifications of human B7-H2 gene expression can be obtained by designing antisense oligonucleotides which will form duplexes to the control, 5′, or regulatory regions of the human B7-H2 gene. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (e.g., Gee et al., in Huber & Carr, MOLECULAR AND IMMUNOLOGIC APPROACHES, Futura Publishing Co., Mt. Kisco, N.Y., 1994). An antisense oligonucleotide also can be designed to block translation of mRNA by preventing the transcript from binding to ribosomes.

Precise complementarity is not required for successful complex formation between an antisense oligonucleotide and the complementary sequence of a human B7-H2 polynucleotide. Antisense oligonucleotides which comprise, for example, 2, 3, 4, or 5 or more stretches of contiguous nucleotides which are precisely complementary to a human B7-H2 polynucleotide, each separated by a stretch of contiguous nucleotides which are not complementary to adjacent human B7-H2 nucleotides, can provide sufficient targeting specificity for human B7-H2 mRNA. Preferably, each stretch of complementary contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular human B7-H2 polynucleotide sequence.

Antisense oligonucleotides can be modified without affecting their ability to hybridize to a human B7-H2 polynucleotide. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′, 5′-substituted oligonucleotide in which the 3′ hydroxyl group or the 5′ phosphate group are substituted, also can be employed in a modified antisense oligonucleotide. These modified oligonucleotides can be prepared by methods well known in the art. See, e.g., Agrawal et al., Trends Biotechnol. 10, 152-158, 1992; Uhlmann et al., Chem. Rev. 90, 543-584, 1990; Uhlmann et al., Tetrahedron Lett. 215, 3539-3542, 1987.

### Ribozymes

Ribozymes are RNA molecules with catalytic activity. See, e.g., Cech, Science 236, 1532-1539; 1987; Cech, Ann. Rev. Biochem. 59, 543-568; 1990, Cech, Curr. Opin. Struct. Biol. 2, 605-609; 1992, Couture & Stinchcomb, Trends Genet. 12, 510-515, 1996. Ribozymes can be used to inhibit gene function by cleaving an RNA sequence, as is known in the art (e.g., Haseloff et al., U.S. Pat. No. 5,641,673). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences.

The coding sequence of a human B7-H2 polynucleotide can be used to generate ribozymes which will specifically bind to mRNA transcribed from the human B7-H2 polynucleotide. Methods of designing and constructing ribozymes which can cleave other RNA molecules in trans in a highly sequence specific manner have been developed and described in the art (see Haseloff et al. Nature 334, 585-591, 1988). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach et al., EP 321,201).

Specific ribozyme cleavage sites within a human B7-H2 RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate human B7-H2 RNA targets also can be evaluated by testing accessibility to hybridization with complementary oligonucleotides using ribonuclease protection assays. Longer complementary sequences can be used to increase the affinity of the hybridization sequence for the target The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease human B7-H2 expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or UAS element, and a transcriptional terminator signal, for controlling transcription of ribozymes in the cells.

As taught in Haseloff et al., U.S. Pat. No. 5,641,673, ribozymes can be engineered so that ribozyme expression will occur in response to factors which induce expression of a target gene. Ribozymes also can be engineered to provide an additional level of regulation, so that destruction of mRNA occurs only when both a ribozyme and a target gene are induced in the cells.

Differentially Expressed Genes

Described herein are methods for the identification of genes whose products interact with human B7-H2. Such genes may represent genes which are differentially expressed in disorders including, but not limited to, autoimmune diseases, allergic diseases, bacterial infections, and type I diabetes. Further, such genes may represent genes which are differentially regulated in response to manipulations relevant to the progression or treatment of such diseases. Additionally, such genes may have a temporally modulated expression, increased or decreased at different stages of tissue or organism development. A differentially expressed gene may also have its expression modulated under control versus experimental conditions. In addition, the human B7-H2 gene or gene product may itself be tested for differential expression.

The degree to which expression differs in a normal versus a diseased state need only be large enough to be visualized via standard characterization techniques such as differential display techniques. Other such standard characterization techniques by which expression differences may be visualized include but are not limited to, quantitative RT (reverse transcriptase), PCR, and Northern analysis.

Identification of Differentially Expressed Genes

To identify differentially expressed genes total RNA or, preferably, mRNA is isolated from tissues of interest. For example, RNA samples are obtained from tissues of experimental subjects and from corresponding tissues of control subjects. Any RNA isolation technique which does not select against the isolation of mRNA may be utilized for the purification of such RNA samples. See, for example, Ausubel et al., ed., CURRENT PROTOCOLS IN MOLECULAR BIOLOGY, John Wiley & Sons, Inc. New York, 1987-1993. Large numbers of tissue samples may readily be processed using techniques well known to those of skill in the art, such as, for example, the single-step RNA isolation process of Chomczynski, U.S. Pat. No. 4,843,155.

Transcripts within the collected RNA samples which represent RNA produced by differentially expressed genes are identified by methods well known to those of skill in the art. They include, for example, differential screening (Tedder et al., Proc. Natl. Acad. Sci. USA. 85, 208-12, 1988), subtractive hybridization (Hedrick et al., Nature 308, 149-53; Lee et al., Proc. Natl. Acad. Sci. USA. 88, 2825, 1984), and, preferably, differential display (Liang & Pardee, Science 257, 967-71, 1992; U.S. Pat. No. 5,262,311).

The differential expression information may itself suggest relevant methods for the treatment of disorders involving the human B7-H2. For example, treatment may include a modulation of expression of the differentially expressed genes and/or the gene encoding the human B7-H2. The differential expression information may indicate whether the expression or activity of the differentially expressed gene or gene product or the human B7-H2 gene or gene product are up-regulated or down-regulated.

Screening Methods

The invention provides assays for screening test compounds which bind to or modulate the activity of a human B7-H2 polypeptide or a human B7-H2 polynucleotide. A test compound preferably binds to a human B7-H2 polypeptide or polynucleotide. More preferably, a test compound decreases or increases human B7-H2 activity by at least about 10, preferably about 50, more preferably about 75, 90, or 100% relative to the absence of the test compound.

Test Compounds

Test compounds can be pharmacologic agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries, while the other four approaches are applicable to polypeptide, non-peptide oligomer, or small molecule libraries of compounds. See Lam, Anticancer Drug Des. 12, 145, 1997.

Methods for the synthesis of molecular libraries are well known in the art (see, for example, DeWitt et al., Proc. Natl. Acad. Sci. USA. 90, 6909, 1993; Erb et al. Proc. Natl. Acad. Sci. U.S.A. 91, 11422, 1994; Zuckermann et al., J. Med Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed Engl. 33, 2061; Gallop et al., J. Med. Chem. 37, 1233, 1994). Libraries of compounds can be presented in solution (see, e.g., Houghten, BioTechniques 13, 412-421, 1992), or on beads (Lan, Nature 354, 82-84, 1991), chips (Fodor, Nature 364, 555-556, 1993), bacteria or spores (Ladner, U.S. Pat. No. 5,223,409), plasmids (Cull et al., Proc. Natl. Acad. Sci. U.S.A. 89, 1865-1869, 1992), or phage (Scott & Smith, Science 249, 386-390, 1990; Devlin, Science 249, 404-406, 1990); Cwirla et al., Proc. Natl. Acad. Sci. 97, 6378-6382, 1990; Felici, J. Mol. Biol. 222, 301-310, 1991; and Ladner, U.S. Pat. No. 5,223,409).

High Throughput Screening

Test compounds can be screened for the ability to bind to human B7-H2 polypeptides or polynucleotides or to affect human B7-H2 activity or human B7-H2 gene expression using high throughput screening. Using high throughput screening, many discrete compounds can be tested in parallel so that large numbers of test compounds can be quickly screened. The most widely established techniques utilize 96-well microtiter plates. The wells of the microtiter plates typically require assay volumes that range from 50 to 500 μl. In addition to the plates, many instruments, materials, pipettors, robotics, plate washers, and plate readers are commercially available to fit the 96-well format.

Alternatively, “free format assays,” or assays that have no physical barrier between samples, can be used. For example, an assay using pigment cells (melanocytes) in a simple homogeneous assay for combinatorial peptide libraries is described by Jayawickreme et al., Proc. Natl. Acad. Sci. U.S.A. 19, 1614-18 (1994). The cells are placed under agarose in petri dishes, then beads that carry combinatorial compounds are placed on the surface of the agarose. The combinatorial compounds are partially released the compounds from the beads. Active compounds can be visualized as dark pigment areas because, as the compounds diffuse locally into the gel matrix, the active compounds cause the cells to change colors.

Another example of a free format assay is described by Chelsky, “Strategies for Screening Combinatorial Libraries: Novel and Traditional Approaches,” reported at the First Annual Conference of The Society for Biomolecular Screening in Philadelphia, Pa (Nov. 7-10, 1995). Chelsky placed a simple homogenous enzyme assay for carbonic anhydrase inside an agarose gel such that the enzyme in the gel would cause a color change throughout the gel. Thereafter, beads carrying combinatorial compounds via a photolinker were placed inside the gel and the compounds were partially released by UV-light. Compounds that inhibited the enzyme were observed as local zones of inhibition having less color change.

Yet another example is described by Salmon et al., Molecular Diversity 2, 57-63 (1996). In this example, combinatorial libraries were screened for compounds that had cytotoxic effects on cancer cells growing in agar.

Another high throughput screening method is described in Beutel et al., U.S. Pat. No. 5,976,813. In this method, test samples are placed in a porous matrix. One or more assay components are then placed within, on top of, or at the bottom of a matrix such as a gel, a plastic sheet, a filter, or other form of easily manipulated solid support. When samples are introduced to the porous matrix they diffuse sufficiently slowly, such that the assays can be performed without the test samples running together.

Binding Assays

For binding assays, the test compound is preferably a small molecule which binds to and occupies, for example, the active site of the human B7-H2 polypeptide, such that normal biological activity is prevented. Examples of such small molecules include, but are not limited to, small peptides or peptide-like molecules.

In binding assays, either the test compound or the human B7-H2 polypeptide can comprise a detectable label, such as a fluorescent, radioisotopic, chemiluminescent, or enzymatic label, such as horseradish peroxidase, alkaline phosphatase, or luciferase. Detection of a test compound which is bound to the human B7-H2 polypeptide can then be accomplished, for example, by direct counting of radio-emmission, by scintillation counting, or by determining conversion of an appropriate substrate to a detectable product

Alternatively, binding of a test compound to a human B7-H2 polypeptide can be determined without labeling either of the interactants. For example, a microphysiometer can be used to detect binding of a test compound with a human B7-H2 polypeptide. A microphysiometer (e.g., Cytosensor™) is an analytical instrument that measures the rate at which a cell acidifies its environment using a light-addressable potentiometric sensor (LAPS). Changes in this acidification rate can be used as an indicator of the interaction between a test compound and a human B7-H2 polypeptide (McConnell et al., ^{Science} 257, 1906-1912, 1992).

Determining the ability of a test compound to bind to a human B7-H2 polypeptide also can be accomplished using a technology such as real-time Bimolecular Interaction Analysis (BIA) (Sjolander & Urbaniczky, Anal. Chem. 63, 2338-2345, 1991, and Szabo et al., Curr. Opin. Struct. Biol. 5, 699-705, 1995). BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

In yet another aspect of the invention, a human B7-H2 polypeptide can be used as a “bait protein” in a two-hybrid assay or three-hybrid assay (see, e.g., U.S. Pat. No. 5,283,317; Zervos et al., Cell 72, 223-232, 1993; Madura et al., J. Biol. Chem. 268, 12046-12054, 1993; Bartel et al., BioTechniques 14, 920-924, 1993; Iwabuchi et al., Oncogene 8, 1693-1696, 1993; and Brent WO94/10300), to identify other proteins which bind to or interact with the human B7-H2 polypeptide and modulate its activity.

The two-hybrid system is based on the modular nature of most transcription factors, which consist of separable DNA-binding and activation domains. Briefly, the assay utilizes two different DNA constructs. For example, in one construct, polynucleotide encoding a human B7-H2 polypeptide can be fused to a polynucleotide encoding the DNA binding domain of a known transcription factor (e.g., GAL-4). In the other construct a DNA sequence that encodes an unidentified protein (“prey” or “sample”) can be fused to a polynucleotide that codes for the activation domain of the known transcription factor. If the “bait” and the “prey” proteins are able to interact in vivo to form an protein-dependent complex, the DNA-binding and activation domains of the transcription factor are brought into close proximity. This proximity allows transcription of a reporter gene (e.g., LacZ), which is operably linked to a transcriptional regulatory site responsive to the transcription factor. Expression of the reporter gene can be detected, and cell colonies containing the functional transcription factor can be isolated and used to obtain the DNA sequence encoding the protein which interacts with the human B7-H2 polypeptide.

It may be desirable to immobilize either the human B7-H2 polypeptide (or polynucleotide) or the test compound to facilitate separation of bound from unbound forms of one or both of the interactants, as well as to accommodate automation of the assay. Thus, either the human B7-H2 polypeptide (or polynucleotide) or the test compound can be bound to a solid support. Suitable solid supports include, but are not limited to, glass or plastic slides, tissue culture plates, microtiter wells, tubes, silicon chips, or particles such as beads (including, but not limited to, latex, polystyrene, or glass beads). Any method known in the art can be used to attach the enzyme polypeptide (or polynucleotide) or test compound to a solid support, including use of covalent and non-covalent linkages, passive absorption, or pairs of binding moieties attached respectively to the polypeptide (or polynucleotide) or test compound and the solid support. Test compounds are preferably bound to the solid support in an array, so that the location of individual test compounds can be tracked Binding of a test compound to a human B7-H2 polypeptide (or polynucleotide) can be accomplished in any vessel suitable for containing the reactants. Examples of such vessels include microtiter plates, test tubes, and microcentrifuge tubes.

In one embodiment, the human B7-H2 polypeptide is a fusion protein comprising a domain that allows the human B7-H2 polypeptide to be bound to a solid support. For example, glutathione-S-transferase fusion proteins can be adsorbed onto glutathione sepharose beads (Sigma Chemical, St. Louis, Mo.) or glutathione derivatized microtiter plates, which are then combined with the test compound or the test compound and the non-adsorbed human B7-H2 polypeptide; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined.

Other techniques for immobilizing proteins or polynucleotides on a solid support also can be used in the screening assays of the invention. For example, either a human B7-H2 polypeptide (or polynucleotide) or a test compound can be immobilized utilizing conjugation of biotin and streptavidin. Biotinylated human B7-H2 polypeptides (or polynucleotides) or test compounds can be prepared from biotin-NHS(N-hydroxysuccinimide) using techniques well known in the art (e.g., biotinylation kit, Pierce Chemicals, Rockford, Ill.) and immobilized in the wells of streptavidin-coated 96 well plates (Pierce Chemical). Alternatively, antibodies which specifically bind to a human B7-H2 polypeptide, polynucleotide, or a test compound, but which do not interfere with a desired binding site, such as the active site of the human B7-H2 polypeptide, can be derivatized to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to the human B7-H2 polypeptide or test compound, enzyme-linked assays which rely on detecting an activity of the human B7-H2 polypeptide, and SDS gel electrophoresis under non-reducing conditions.

Screening for test compounds which bind to a human B7-H2 polypeptide or polynucleotide also can be carried out in an intact cell. Any cell which comprises a human B7-H2 polypeptide or polynucleotide can be used in a cell-based assay system. A human B7-H2 polynucleotide can be naturally occurring in the cell or can be introduced using techniques such as those described above. Binding of the test compound to a human B7-H2 polypeptide or polynucleotide is determined as described above.

Gene Expression

In another embodiment, test compounds which increase or decrease human B7-H2 gene expression are identified. A human B7-H2 polynucleotide is contacted with a test compound, and the expression of an RNA or polypeptide product of the human B7-H2 polynucleotide is determined The level of expression of appropriate mRNA or polypeptide in the presence of the test compound is compared to the level of expression of mRNA or polypeptide in the absence of the test compound. The test compound can then be identified as a modulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression.

The level of human B7-H2 mRNA or polypeptide expression in the cells can be determined by methods well known in the art for detecting mRNA or polypeptide. Either qualitative or quantitative methods can be used. The presence of polypeptide products of a human B7-H2 polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into a human B7-H2 polypeptide.

Such screening can be carried out either in a cell-free assay system or in an intact cell. Any cell which expresses a human B7-H2 polynucleotide can be used in a cell-based assay system. The human B7-H2 polynucleotide can be naturally occurring in the cell or can be introduced using techniques such as those described above. Either a primary culture or an established cell line, such as CHO or human embryonic kidney 293 cells, can be used.

Pharmaceutical Compositions

The invention also provides pharmaceutical compositions that can be administered to a patient to achieve a therapeutic effect. Pharmaceutical compositions of the invention can comprise, for example, a human B7-H2 polypeptide, human B7-H2 polynucleotide, ribozymes or antisense oligonucleotides, antibodies which specifically bind to a human B7-H2 polypeptide, or mimetics, activators, or inhibitors of a human B7-H2 polypeptide activity. The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones.

In addition to the active ingredients, these pharmaceutical compositions can contain suitable pharmaceutically-acceptable carriers comprising excipients and auxiliaries that facilitate processing of the active compounds into preparations which can be used pharmaceutically. Pharmaceutical compositions of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents can be added, such as the cross-linked polyvinyl pyrrolidone, agar, alginic acid, or a salt thereof, such as sodium alginate.

Dragee cores can be used in conjunction with suitable coatings, such as concentrated sugar solutions, which also can contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound, ie., dosage.

Pharmaceutical preparations that can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances that increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers also can be used for delivery. Optionally, the suspension also can contain suitable stabilizers or agents that increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee-making, levigating, emulsifying, encapsulating, entrapping, or lyophilizing processes. The pharmaceutical composition can be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation can be a lyophilized powder which can contain any or all of the following: 1-50 mM histidine, 0.1%-2% sucrose, and 2-7% mannitol, at a pH range of 4.5 to 5.5, that is combined with buffer prior to use.

Further details on techniques for formulation and administration can be found in the latest edition of REMINGTON'S PHARMACEUTICAL SCIENCES (Maack Publishing Co., Easton, Pa). After pharmaceutical compositions have been prepared, they can be placed in an appropriate container and labeled for treatment of an indicated condition. Such labeling would include amount, frequency, and method of administration.

Therapeutic Indications and Methods

Human B7-H2 protein may be regulated to treat autoimmune diseases, allergic diseases, bacterial infections, and type I diabetes.

In order for the body to defend itself against different pathogens, different types of immune responses are necessary. For some pathogens, such as intracellular bacteria, a predominantly Th1 type of response is required to control infection, while for others, such as helminths or microbes presesnt in the extracellular milieu, a Th2 type of response is required. Inappropriate polarization of immune responses can result in inadequate protection against infection, while unregulated overpolarization of responses can have harmful sequelae. In the case of intracellular bacterial infections, the counterregulatory cytokine IL-1 is secreted rapidly after infection to control Th1 responses (ref 12). Such a response rely on B7-H2 both to transmit signals into the cell on which it is expressed and to stimulate ICOS on T cells. Similarly, when a Th2 response is appropriate, the amplification of the response by signaling through B7-H2 and stimulation of ICOS is necessary for adequate defense against a pathogen. On the other hand, in autoimmune diseases and allergic diseases, uncontrolled activation of the immune response causes tissue distruction, suffering, and sometimes life-threatening complications. Upregulated expression of B7-H2 following Th1 immune responses and downregulated expression of B7-H2 following Th2 immune responses is a possible method that the body can use to avoid autoimmunity and allergy after normal immune responses. The expression of different splice variants of B7-H2 also allows different cells to respond differently according to the situation. Therefore a cell's decision on which B7-H2 variant to express and at what level may be crucial to the development and control of an appropriate immune response.

The development of inhibitors to block the functions of the B7-H2 V1 and B7-H2 V2 would be expected to be useful in the treatment of allergic diseases, such as respiratory allergies, food allergies, asthma, and atopic dermatitis, as well as in the treatment of intracellular bacterial infections, such as tuberculosis, leprosy, listeriosis, and salmonellosis, where a downregulation of the Th2 response and a repolarization towards a Th1 response would be beneficial. The development of molecules to enhance the functions of B7-H2 VI and B7-H2 V2 is useful in the treament of autoimmune diseases, such as multiple sclerosis, rheumatoid arthritis, and type I diabetes, as well as in the treatment of helminth and extracellular microbial infections, where a repolarization towards a Th2 response would be beneficial.

This invention further pertains to the use of novel agents identified by the screening assays described above. Accordingly, it is within the scope of this invention to use a test compound identified as described herein in an appropriate animal model. For example, an agent identified as described herein (e.g., a modulating agent, an antisense nucleic acid molecule, a specific antibody, ribozyme, or a human B7-H2 polypeptide binding molecule) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

A reagent which affects human B7-H2 activity can be administered to a human cell, either in vitro or in vivo, to reduce human B7-H2 activity. The reagent preferably binds to an expression product of a human B7-H2 gene. If the expression product is a protein, the reagent is preferably an antibody. For treatment of human cells ex vivo, an antibody can be added to a preparation of stem cells that have been removed from the body. The cells can then be replaced in the same or another human body, with or without clonal propagation, as is known in the art.

In one embodiment, the reagent is delivered using a liposome. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human. Preferably, the lipid composition of the liposome is capable of targeting to a specific organ of an animal, such as the lung, liver, spleen, heart brain, lymph nodes, and skin.

A liposome useful in the present invention comprises a lipid composition that is capable of fusing with the plasma membrane of the targeted cell to deliver its contents to the cell. Preferably, the transfection efficiency of a liposome is about 0.5 μg of DNA per 16 nmole of liposome delivered to about 10⁶ cells, more preferably about 1.0 μg of DNA per 16 nmole of liposome delivered to about 10⁶ cells, and even more preferably about 2.0 μg of DNA per 16 nmol of liposome delivered to about 10⁶ cells. Preferably, a liposome is between about 100 and 500 nm, more preferably between about 150 and 450 nm, and even more preferably between about 200 and 400 nm in diameter.

Suitable liposomes for use in the present invention include those liposomes standardly used in, for example, gene delivery methods known to those of skill in the art. More preferred liposomes include liposomes having a polycationic lipid composition and/or liposomes having a cholesterol backbone conjugated to polyethylene glycol. Optionally, a liposome comprises a compound capable of targeting the liposome to a particular cell type, such as a cell-specific ligand exposed on the outer surface of the liposome.

Complexing a liposome with a reagent such as an antisense oligonucleotide or ribozyme can be achieved using methods that are standard in the art (see, for example, U.S. Pat. No. 5,705,151). Preferably, from about 0.1 μg to about 10 μg of polynucleotide is combined with about 8 nmol of liposomes, more preferably from about 0.5 μg to about 5 μg of polynucleotides are combined with about 8 nmol liposomes, and even more preferably about 1.0 μg of polynucleotides is combined with about 8 nmol liposomes.

In another embodiment, antibodies can be delivered to specific tissues in vivo using receptor-mediated targeted delivery. Receptor-mediated DNA delivery techniques are taught in, for example, Findeis et al. Trends in Biotechnol. 11, 202-05 (1993); Chiou et al., GENE THERAPEUTICS: METHODS AND APPLICATIONS OF DIRECT GENE TRANSFER (J. A. Wolff, ed.) (1994); Wu & Wu, J. Biol. Chem. 263, 621-24 (1988); Wu et al., J. Biol. Chem. 269, 542-46 (1994); Zenke et al., Proc. Natl. Acad. Sci. U.S.A. 87, 3655-59 (1990); Wu et al., J. Biol. Chem. 266, 338-42 (1991).

Determination of a Therapeutically Effective Dose

The determination of a therapeutically effective dose is well within the capability of those skilled in the art. A therapeutically effective dose refers to that amount of active ingredient that increases or decreases human B7-H2 activity relative to the human B7-H2 activity which occurs in the absence of the therapeutically effective dose.

For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀.

Pharmaceutical compositions that exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active ingredient or to maintain the desired effect. Factors that can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Normal dosage amounts can vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

If the reagent is a single-chain antibody, polynucleotides encoding the antibody can be constructed and introduced into a cell either ex vivo or in vivo using well-established techniques including, but not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and DEAE- or calcium phosphate-mediated transfection.

Effective in vivo dosages of an antibody are in the range of about 5 μg to about 50 μg/kg, about 50 μg to about 5 mg/kg, about 100 μg to about 500 μg/kg of patient body weight, and about 200 to about 250 μg/kg of patient body weight. For administration of polynucleotides encoding single-chain antibodies, effective in vivo dosages are in the range of about 100 ng to about 200 ng, 500 ng to about 50 mg, about 1 μg to about 2 mg, about 5 μg to about 500 μg, and about 20 μg to about 100 μg of DNA.

If the expression product is mRNA, the reagent is preferably an antisense oligonucleotide or a ribozyme. Polynucleotides that express antisense oligonucleotides or ribozymes can be introduced into cells by a variety of methods, as described above.

Preferably, a reagent reduces expression of a human B7-H2 gene or the activity of a human B7-H2 polypeptide by at least about 10, preferably about 50, more preferably about 75, 90, or 100% relative to the absence of the reagent. The effectiveness of the mechanism chosen to decrease the level of expression of a human B7-H2 gene or the activity of a human B7-H12 polypeptide can be assessed using methods well known in the art, such as hybridization of nucleotide probes to human B7-H2-specific mRNA, quantitative RT-PCR, immunologic detection of a human B7-H2 polypeptide, or measurement of human B7-H2 activity.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects.

Any of the therapeutic methods described above can be applied to any subject in need of such therapy, including, for example, mammals such as dogs, cats, cows, horses, rabbits, monkeys, and most preferably, humans.

Diagnostic Methods

Human B7-H2 also can be used in diagnostic assays for detecting diseases and abnormalities or susceptibility to diseases and abnormalities related to the presence of mutations in the nucleic acid sequences that encode the enzyme. For example, differences can be determined between the cDNA or genomic sequence encoding human B7-H2 in individuals afflicted with a disease and in normal individuals. If a mutation is observed in some or all of the afflicted individuals but not in normal individuals, then the mutation is likely to be the causative agent of the disease.

Sequence differences between a reference gene and a gene having mutations can be revealed by the direct DNA sequencing method. In addition, cloned DNA segments can be employed as probes to detect specific DNA segments. The sensitivity of this method is greatly enhanced when combined with PCR For example, a sequencing primer can be used with a double-stranded PCR product or a single-stranded template molecule generated by a modified PCR. The sequence determination is performed by conventional procedures using radiolabeled nucleotides or by automatic sequencing procedures using fluorescent tags.

Genetic testing based on DNA sequence differences can be carried out by detection of alteration in electrophoretic mobility of DNA fragments in gels with or without denaturing agents. Small sequence deletions and insertions can be visualized, for example, by high resolution gel electrophoresis. DNA fragments of different sequences can be distinguished on denaturing formamide gradient gels in which the mobilities of different DNA fragments are retarded in the gel at different positions according to their specific melting or partial melting temperatures (see, e.g., Myers et al., Science 230, 1242, 1985). Sequence changes at specific locations can also be revealed by nuclease protection assays, such as RNase and S 1 protection or the chemical cleavage method (e.g., Cotton et al., Proc. Natl. Acad. Sci. USA 85, 4397-4401, 1985). Thus, the detection of a specific DNA sequence can be performed by methods such as hybridization, RNase protection, chemical cleavage, direct DNA sequencing or the use of restriction enzymes and Southern blotting of genomic DNA. In addition to direct methods such as gel-electrophoresis and DNA sequencing, mutations can also be detected by in situ analysis.

Altered levels of a human B7-H2 also can be detected in various tissues. Assays used to detect levels of the receptor polypeptides in a body sample, such as blood or a tissue biopsy, derived from a host are well known to those of skill in the art and include radioimmunoassays, competitive binding assays, Western blot analysis, and ELISA assays.

All patents and patent applications cited in this disclosure are expressly incorporated herein by reference. The above disclosure generally describes the present invention. A more complete understanding can be obtained by reference to the following specific examples which are provided for purposes of illustration only and are not intended to limit the scope of the invention.

### EXAMPLE 1

Determination of the Human B7-H2 V1 and V2 N?RNA Sequence

The human amino acid sequences for CD80 (GenBank accession number NP_{—}005182), CD86 (GenBank accession number NP_{—}008820), and B7H1 (GenBank accession number NP_{—}054862), and the mouse mRNA sequence for B7h (GenBank accession number NP_{—}056605) were used to search the DNA DataBank of Japan (DDBJ) for homologous sequences using the TBLASTN component of the computer program BLAST 2.0 (National Center for Biotechnology Information). One human DNA sequence (GenBank accession number AB014553, annotated as the homo sapiens mRNA for KIAA0653 protein) was found that when conceptually translated had over 50% amino acid sequence identity with the mouse B7h amino acid sequence over a region of 231 residues and over 64% amino acid homology over a region of 231 residues.

The predicted open reading frame of the KIAA0653 gene was then cloned for further analysis. Primers flanking the open reading frame were designed using the computer program Primer 3.0 (Steve Rozen, Helen J. Skaletsky (1998) Primer3. Code available at http://www-genome.wi.mit.edu/genome_software/other/primer3.html.). Primers K653-L2 (SEQ ID NO:5) and K653-R6 (SEQ ID NO:6) were used to amplify the open reading by polymerase chain reaction using human peripheral blood leukocyte cDNA as the template in the reaction. The template cDNA was previously synthesized with the SMART RACE cDNA amplification kit (Clontech, Palo Alto, Calif., USA) according to the manufacturer's protocol using human peripheral blood leukocyte-derived poly-A RNA as the starting material for cDNA synthesis. Successfully amplified fragments were cloned into the pCRII-TOPO vector (Invitrogen, Carlsbad, Calif., USA) and were sequenced on a ABI Prism 377 DNA sequencer (PE Biosystems) according to the manufacturer's standard sequencing protocol using primers complemetary to the SP6 and T7 promoter regions flanking the insert on each vector. After sequencing, the DNA sequences of five selected clones (clones 14, 16, 17, 19, and 21) were compared with the published sequence for KIAA0653 using the computer program Sequencher (Gene Codes Corporation, Ann Arbor, Mich., USA).

Properties of the Obtained cDNA)

1. The sequence of transcript 1 (from clone 14) has a coding region 912 bp in length.

2. Compared with the original KIAA0653 mRNA sequence (GenBank accession number AB014553) reported for this gene, transcript 1 has a deletion of 636 bp from bases 1027 to 1662 of the KIAA0653 sequence. In addition, the G nucleotide at position 510 of KIAA0653 is changed to an A position 482) in transcript 1.

3. Compared with the GL50 mRNA sequence (GenBank accession number AF199028) reported for this gene, transcript 1 shows no significant homology in its 3′ end (after base 998) with the 3′ end (after base 921) of the of the GL50 sequence. In addition, the G nucleotide at position 405 of GL50 is changed to an A (position 482) in transcript 1.

4. Compared with the B7-H2 mRNA sequence (GenBank accession number AF289028) reported for this gene, transcript 1 shows no significant homology in its 3′ end (after base 998) with the 3′ end (after base 1022) of the of the B7-H2 sequence. In addition, the G nucleotide at position 506 of B7-H2 is changed to an A (position 482) in transcript 1.

5. The sequence of transcript 2 (from clones 16, 17, 19, and 21) has a coding region 1419 bp in length.

6. Compared with the original KIAA0653 mRNA sequence reported for this gene, transcript 2 has a deletion of 129 bp from bases 1452 to 1580 of the KIAA0653 sequence. In addition, several nucleotide sequence differences outside of the deleted region are noted between transcript 2 and the original KIAA0653 sequence:

KIAA0653 nucleotide 510 changed from G to A (transcript 2 position 482)

KIAA0653 nucleotide 1115 changed from G to A (transcript 2 position 1087)

KIAA0653 nucleotide 1185 changed from C to T (transcript 2 position 1157)

KIAA0653 nucleotide 1323 changed from G to A (transcript 2 position 1295)

KIAA0653 nucleotide 1593 changed from T to C (transcript 2 position 1436)

7. Compared with the GL50 mRNA sequence (GenBank accession number AF199028) reported for this gene, transcript 2 shows no significant homology in its 3′ end (after base 998) with the 3′ end (after base 921) of the of the GL50 sequence. In addition, the G nucleotide at position 405 of GL50 is changed to an A (position 482) in transcript 2.

8. Compared with the B7-H2 mRNA sequence (GenBank accession number AF289028) reported for this gene, transcript 2 shows no significant homology in its 3′ end (after base 998) with the 3′ end (after base 1022) of the of the B7-H2 sequence. In addition, the G nucleotide at position 506 of B7-H2 is changed to an A (position 482) in transcript 2.

(Properties of the Amino Acid Sequences Encoded by the Obtained cDNA)

1. The translation of the transcript 1 clone (B7-H2 V1) gave an amino acid sequence 304 residues in length.

2. The translation of the transcript 1 sequence differs from the conceptual translation of KIAA0653 (GenBank accession number BAA31628) by lacking the first 42 residues of KIAA0653 and having a substitution of valine (KIAA0653 residue 170) to isoleucine (B7-H2 V1 residue 128). KIAA0653 residues 342 through 553, corresponding to bases 1027-1662 of the KIAA0653 transcript, are also lacking.

3. The translation of the transcript 1 sequence differs from the conceptual translation of GL50 (GenBank accession number AAF34739) by having a substitution of valine (GL50 residue 128) to isoleucine (137-H2 V1 residue 128), but is otherwise identical in the first 299 residues. The carboxy terminals after residue 299 of both sequences show no significant homology.

4. The translation of the transcript 1 sequence differs from the conceptual translation of B7-H2 (GenBank accession number AAG01176) by having a substitution of valine (GL50 residue 128) to isoleucine (B7-H2 V1 residue 128), but is otherwise identical in the first 299 residues. The carboxy terminals after residue 299 of both sequences show no significant homology.

5. The translation of the transcript 2 clones gives an amino acid sequence 473 residues in length.

6. The translation of the transcript 2 sequence differs from the conceptual translation of KIAA0653 at the following residues:

KIAA0653 residue 170 valine changed to isoleucine (B7-H2 V2 residue 128)

KIAA0653 residue 395 arginine changed to tryptophan (B7-H2 V2 residue 353)

KIAA0653 residue 441 aspartate changed to asparagine (B7-H2 V2 residue 399)

KIAA0653 residue 531 tryptophan changed to arginine (137-H2 V2 residue 446)

KIAA0653 residues 484 through 526, corresponding to bases 1452-1580 of the K1AA0653

transcript, are deleted.

7. The translation of the transcript 2 sequence differs from the conceptual translation of GL50 (GenBank accession number AAF34739) by having a substitution of valine (GL50 residue 128) to isoleucine (B7-H2 V2 residue 128), but is otherwise identical in the first 299 residues. The carboxy terminals after residue 299 of both sequences show no significant homology.

8. The translation of the transcript 2 sequence differs from the conceptual translation of B7-H2 (GenBank accession number AAG01176) by having a substitution of valine (GL50 residue 128) to isoleucine (B7-H2 V2 residue 128), but is otherwise identical in the first 299 residues. The carboxy terminals after residue 299 of both sequences show no significant homology.

### EXAMPLE 2

Tissue Distribution of Human B7-H2

Expression profiling is based on a quantitative polymerase chain reaction (PCR) analysis, also called kinetic analysis, first described in Higuchi et al., 1992 and Higuchi et al., 1993. The principle is that at any given cycle within the exponential phase of PCR, the amount of product is proportional to the initial number of template copies. Using this technique, the expression levels of particular genes, which are transcribed from the chromosomes as messenger RNA (mRNA), are measured by first making a DNA copy (cDNA) of the mRNA, and then performing quantitative PCR on the cDNA, a method called quantitative reverse transcription-polymerase chain reaction (quantitative RT-PCR).

Quantitative RT-PCR analysis of RNA from different human tissues was performed to investigate the tissue distribution of B7-H2 transcript 1 or 2 mRNA. 25 .mu.g of total RNA from various tissues (Human Total RNA Panel I-V, Clontech Laboratories, Palo Alto, Calif., USA) was used as a template to synthsize first-strand cDNA using the SUPERSCRIPT™ First-Strand Synthesis System for RT-PCR (Life Technologies, Rockville, Md., USA). First-strand cDNA synthesis was carried out according to the manufacturer's protocol using oligo (dT) to hybridize to the 3′ poly A tails of mRNA and prime the synthesis reaction. 10 ng of the first-strand cDNA was then used as template in a polymerase chain reaction. The polymerase chain reaction was performed in a LightCycler (Roche Molecular Biochemicals, Indianapolis, Ind., USA), in the presence of the DNA-binding fluorescent dye SYBR Green I which binds to the minor groove of the DNA double helix, produced only when double-stranded DNA is successfully synthesized in the reaction (Morrison et al., 1998). Upon binding to double-stranded DNA, SYBR Green I emits light that can be quantitatively measured by the LightCycler machine. The polymerase chain reaction was carried out using oligonucleotide primers K653-L5 (SEQ ID NO:7) and K653-R8 (SEQ ID NO:8) and measurements of the intensity of emitted light were taken following each cycle of the reaction when the reaction had reached a temperature of 87 degrees C. Intensities of emitted light were converted into copy numbers of the gene transcript per nanogram of template cDNA by comparison with simultaneously reacted standards of known concentration.

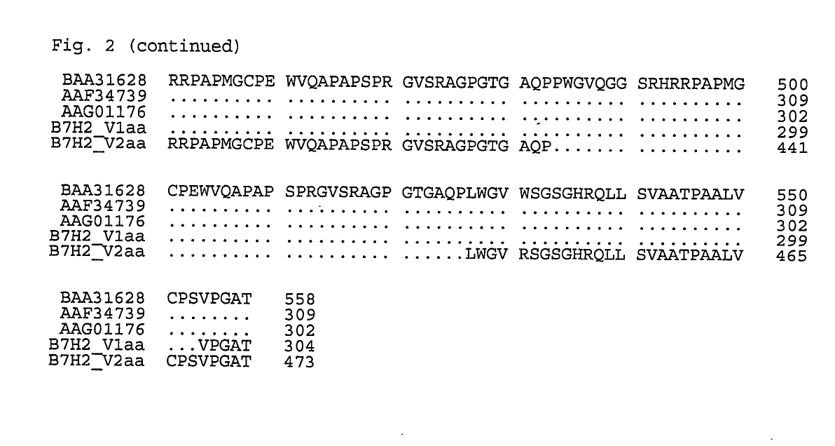

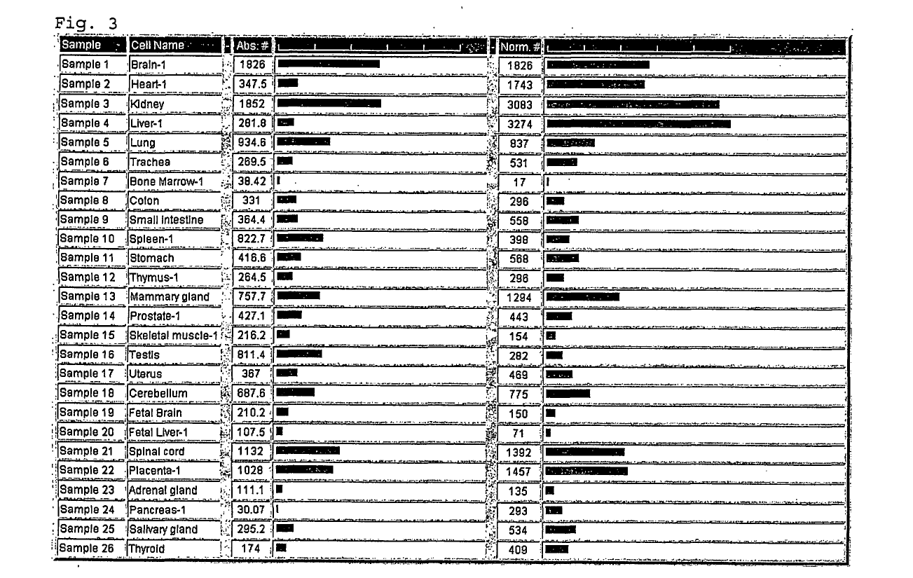

To correct for differences in mRNA transcription levels per cell in the various tissue types, a normalization procedure was performed using similarly calculated expression levels in the various tissues of five different housekeeping genes: glyceraldehyde-3-phosphatase (G3PDH), hypoxanthine guanine phophoribosyl transferase (HPRT), beta-actin, porphobilinogen deaminase (PBGD), and beta-2-microglobulin. The level of housekeeping gene expression is considered to be relatively constant for all tissues (Adams et al., 1993, Adams et al., 1995, Liew et al., 1994) and therefore can be used as a gauge to approximate relative numbers of cells per .mu.g of total RNA used in the cDNA synthesis step. Except for the use of a slightly different set of housekeeping genes and the use of the LightCycler system to measure expression levels, the normalization procedure was essentially the same as that described in the RNA Master Blot User Manual, Apendix C (1997, Clontech Laboratories, Palo Alto, Calif., USA). In brief, expression levels of the five housekeeping genes in all tissue samples were measured in three independent reactions per gene using the LightCycler and a constant amount (25 .mu.g) of starting RNA. The calculated copy numbers for each gene, derived from comparison with simultaneously reacted standards of known concentrations, were recorded and converted into a percentage of the sum of the copy numbers of the gene in all tissue samples. Then for each tissue sample, the sum of the percentage values for each gene was calculated, and a normalization factor was calculated by dividing the sum percentage value for each tissue by the sum percentage value of one of the tissues arbitrarily selected as a standard. To normalize an experimentally obtained value for the expression of a particular gene in a tissue sample, the obtained value was multiplied by the normalization factor for the tissue tested. Results are given in FIG. 3, showing the experimentally obtained copy numbers of mRNA per 10 ng of first-strand cDNA on the left and the normalized values on the right. RNAs used for the cDNA synthesis, along with their supplier and catalog numbers are shown in table 1.

TABLE 1
Table 1. Whole-body-screen tissues

| Tissue | Supplier | Panel name and catalog number |
|---|---|---|
| 1. brain | Clontech | Human Total RNA Panel I, K4000-1 |
| 2. heart | Clontech | Human Total RNA Panel I, K4000-1 |
| 3. kidney | Clontech | Human Total RNA Panel I, K4000-1 |
| 4. liver | Clontech | Human Total RNA Panel I, K4000-1 |
| 5. lung | Clontech | Human Total RNA Panel I, K4000-1 |
| 6. trachea | Clontech | Human Total RNA Panel I, K4000-1 |
| 7. bone marrow | Clontech | Human Total RNA Panel II, K4001-1 |
| 8. colon | Clontech | Human Total RNA Panel II, K4001-1 |
| 9. small intestine | Clontech | Human Total RNA Panel II, K4001-1 |
| 10. spleen | Clontech | Human Total RNA Panel II, K4001-1 |
| 11. stomach | Clontech | Human Total RNA Panel II, K4001-1 |
| 12. thymus | Clontech | Human Total RNA Panel II, K4001-1 |
| 13. mammary gland | Clontech | Human Total RNA Panel III, K4002-1 |
| 14. skeletal muscle | Clontech | Human Total RNA Panel III, K4002-1 |
| 15. prostate | Clontech | Human Total RNA Panel III, K4002-1 |
| 16. testis | Clontech | Human Total RNA Panel III, K4002-1 |
| 17. uterus | Clontech | Human Total RNA Panel III, K4002-1 |
| 18. cerebellum | Clontech | Human Total RNA Panel IV, K4003-1 |
| 19. fetal brain | Clontech | Human Total RNA Panel IV, K4003-1 |
| 20. fetal liver | Clontech | Human Total RNA Panel IV, K4003-1 |
| 21. spinal cord | Clontech | Human Total RNA Panel IV, K4003-1 |
| 22. placenta | Clontech | Human Total RNA Panel IV, K4003-1 |
| 23. adrenal gland | Clontech | Human Total RNA Panel V, K4004-1 |
| 24. pancreas | Clontech | Human Total RNA Panel V, K4004-1 |
| 25. salivary gland | Clontech | Human Total RNA Panel V, K4004-1 |
| 26. thyroid | Clontech | Human Total RNA Panel V, K4004-1 |

As shown in FIG. 3, B7-H2 are broadly expressed in all tissue types so far tested, with highest expression seen in liver, kidney, brain, heart, placenta, spinal cord, mammary gland, and lung.

### EXAMPLE 3

Expression of Human B7-H2

The expression vector pcDNA 3.1 vector Invitrogen, Carlsbad, Calif.) is used to produce large quantities of recombinant human B7-H2 like polypeptides in Chinese hamster ovary (CHO) cells. The human B7-H2-encoding DNA sequence is derived from SEQ ID NO:1 or 2. Before insertion into vector pcDNA 3.1, the DNA sequence is modified by well known methods in such a way that it contains B7-H2 and Ig fusion gene by fusing the cDNA of the extracellular domain of B7-H2 in frame to the CH2-CH3 portion of human IgG1. Moreover, at both termini recognition sequences for restriction endonucleases are added and after digestion of the multiple cloning site of pcDNA 3.1 with the corresponding restriction enzymes the modified DNA sequence is ligated into pcDNA3.1. The resulting phB7-H2 Ig vector is used to transfect the CHO cell, a B7-H2 negative cell line.

The cells are cultivated under usual conditions in 5 liter shake flasks and the secreted recombinantly produced protein (B7-H2 Ig) is purified and used in the next example.

### EXAMPLE 4

T Cell Proliferation with the Costimulation of B7-H2

T cells are purified from human PBMC of healthy donors and then stimulated with B7-H2 Ig obtained in Example 3 in the presence of suboptimal doses of an anti-CD3 mAb. T-cell proliferation is determined by incorporation of ³H-TdR after 3-day culture. B7-H2 Ig enhances T-cell proliferation compared to the control Ig in the presence of immobilized anti-CD3 mAb.

### EXAMPLE 5

Cytokine Secretion by B7-H2 Costimulation

The level of Cytokine e.g., IL-2, IL4, and IL-10 in the T-cell culture supernatants by the stimulation of B7-H21g and an optimal dose of an anti-CD3 mAb are determined by sandwich ELISA. T-cells costimulated by B7-H21g in the presence of an optimal dose of anti-CD3 mAb increase levels of IL-4 and IL-10.

### EXAMPLE 6

Expression of Human ICOS

The expression vector pcDNA 3.1 vector (Invitrogen, Carlsbad, Calif.) is used to produce large quantities of recombinant human ICOS polypeptides in Chinese hamster ovary (CHO) cells. The human ICOS-encoding DNA sequence is derived from the sequence of GenBank accession number AB0231353.

Before insertion into vector pcDNA 3.1, the DNA sequence is modified by well known methods in such a way that it contains ICOS and Ig fusion gene by fusing the cDNA of the extracellular domain of ICOS in frame to the CH2-CH3 portion of human IgG1. Moreover, at both termini recognition sequences for restriction endonucleases are added and after digestion of the multiple cloning site of pcDNA 3.1 with the corresponding restriction enzymes the modified DNA sequence is ligated into pcDNA3.1. The resulting phICOS Ig vector is used to transfect the CHO cell.

The cells are cultivated under usual conditions in 5 liter shake flasks and the secreted recombinantly produced protein (ICOS Ig) is purified and used in the next example.

### EXAMPLE 7

B Cell Proliferation with the Costimulation of ICOS

The expression vector pcDNA 3.1 vector (Invitrogen, Carlsbad, Calif.) is used to produce recombinant human B7-H2 V1 and B7-H2 V2 polypeptides in B-cells. The human B7-H2 V1 and B7-H2 V2 DNA sequence is derived from the sequence of SEQ ID NO:1 and SEQ ID NO:2, respectively.

Before insertion into vector pcDNA 3.1, each of the DNA sequences is modified by well known methods in such a way that it contains at its 5′-end an initiation codon and at its 3′-end a termination codon. Moreover, at both termini recognition sequences for restriction endonucleases are added and after digestion of the multiple cloning site of pcDNA 3.1 with the corresponding restriction enzymes the modified DNA sequence is ligated into pcDNA3.1. The resulting phB7-H2 V1 vector or phB7-H2 V2 is used to transfect the B cell purified from human PBMC of healthy donors.

The cells are cultivated under usual conditions in 5 liter shake flasks and the transfectants with recombinantly produced protein (B7-H2 VI or B7-H2 V2) are obtained. B cells so obtained are then stimulated with ICOS Ig obtained in Example 6 in the presence of suboptimal doses of an anti-CD3 mAb. B-cell proliferation is determined by incorporation of ³H-TdR after 3-day culture. ICOS Ig enhances B-cell proliferation compared to the control Ig in the presence of immobilized anti-CD3 mAb.

### EXAMPLE 8

Cytokine Secretion by ICOS Costimulation

The level of Cytokine e.g., IL-2, IL-4, and IL-10 in the transfectant B-cell culture supernatants by the stimulation of ICOS Ig and an optimal dose of an anti-CD3 mAb are determined by sandwich ELISA. B-cells costimulated by ICOSIg in the presence of an optimal dose of an anti-CD3 mAb change levels of some cytokines.

### EXAMPLE 9

Identification of Test Compounds that Bind to Human B7-H2 Polypeptides

Purified human B7-H2 polypeptides comprising a glutathione-S-transferase protein and absorbed onto glutathione-derivatized wells of 96-well microtiter plates are contacted with test compounds from a small molecule library at pH 7.0 in a physiological buffer solution. Human B7-H2 polypeptides comprise the amino acid sequence shown in SEQ ID NO:2. The test compounds comprise a fluorescent tag. The samples are incubated for 5 minutes to one hour. Control samples are incubated in the absence of a test compound.

The buffer solution containing the test compounds is washed from the wells. Binding of a test compound to a human B7-H2 polypeptide is detected by fluorescence measurements of the contents of the wells. A test compound that increases the fluorescence in a well by at least 15% relative to fluorescence of a well in which a test compound is not incubated is identified as a compound which binds to a human B7-H2 polypeptide.

### EXAMPLE 10

Identification of a Test Compound which Modulates Human B7-H2 Gene Expression

A test compound is administered to a culture of human cells transfected with a human B7-H2 expression construct and incubated at 37° C. for 10 to 45 minutes. A culture of the same type of cells that have not been transfected is incubated for the same time without the test compound to provide a negative control.

RNA is isolated from the two cultures as described in Chirgwin et al., Biochem. 18, 5294-99, 1979). Northern blots are prepared using 20 to 30 μg total RNA and hybridized with a ³²P-labeled human B7-H2-specific probe at 65° C. in Express-hyb (CLONTECH). The probe comprises at least 11 contiguous nucleotides selected from the complement of SEQ ID NO:1 or 2. A test compound that decreases the human B7-H2-specific signal relative to the signal obtained in the absence of the test compound is identified as an inhibitor of human B7-H2 gene expression.

### REFERENCES

1. Ishikawa, K., Nagase, T., Suyama, M., Miyajima, N., Tanaka, A., Kotani, H., Nomura, N. and Ohara, O. Prediction of the coding sequences of unidentified human genes. X. The complete sequences of 100 new cDNA clones from brain which can code for large proteins in vitro. DNA Res. 5 (3), 169-176 (1998).

2. Ling V, Wu P W, Finnerty H F, Bean K M, Spaulding V, Fouser L A, Leonard J P, Hunter S E, Zollner R, Thomas J L, Miyashiro J S, Jacobs K A, Collins M. Cutting edge: identification of GL50, a novel B7-like protein that functionally binds to ICOS receptor. J. Immunol. 2000 Feb. 15;164(4):1653-7.

3. Mages H W, Hutloff A, Heuck C, Buchner K, Himmelbauer H, Oliveri F, Kroczek R A. Molecular cloning and characterization of murine ICOS and identification of B7h as ICOS ligand. Eur J Immunol. 2000 April;30(4):1040-7.

4. Yoshinaga S K, Zhang M, Pistillo J, Horan T, Khare S D, Miner K, Sonnenberg M, Boone T, Brankow D, Dai T, Delaney J, Han H, Hui A, Kohno T, Manoukian R, Whoriskey J S, Coccia M A. Characterization of a new human B7-related protein: B7RP-1 is the ligand to the co-stimulatory protein ICOS. Int Immunol. 2000 October;12(10):1439-47.

5. Yoshinaga S K, Whoriskey J S, Khare S D, Sarmiento U, Guo J, Horan T, Shih G, Zhang M, Coccia M A, Kohno T, Tafuri-Bladt A, Brankow D, Campbell P, Chang D, Chiu L, Dai T, Duncan G, Elliott G S, Hui A, McCabe S M, Scully S, Shahinian A, Shaklee C L, Van G, Mak T W, Senaldi, G. T-cell co-stimulation through B7RP-1 and ICOS. Nature. 1999 Dec. 16;402(6763):827-32.

6. Yoshinaga S K, Zhang M, Pistillo J, Horan T, Khare S D, Miner K, Sonnenberg M, Boone T, Brankow D, Dai T, Delaney J, Han H, Hui A, Kohno T, Manoukian R, Whoriskey J S, Coccia M A. Characterization of a new human B7-related protein: B7RP-1 is the ligand to the co-stimulatory protein ICOS. Int Immunol. 2000 October;12(10):1439-47.

7. Wang S, Zhu G, Chapoval A I, Dong H, Tamada K, Ni J, Chen L. Costimulation of T cells by B7-H2, a B7-like molecule that binds ICOS. Blood. 2000 Oct. 15;96(8):2808-13.

8. Hutloff A, Dittrich A M, Beier K C, Eljaschewitsch B, Kraft R, Anagnostopoulos I, Kroczek R A. ICOS is an inducible T-cell co-stimulator structurally and functionally related to CD28. Nature. 1999 Jan. 21;397(6716):263-6.

9. McAdam A J, Chang T T, Lumelsky A E, Greenfield E A, Boussiotis V A, Duke-Cohan J S, Chernova T, Malenkovich N, Jabs C, Kuchroo V K, Ling V, Collins M, Sharpe A H, Freeman G J. Mouse inducible costimulatory molecule (ICOS) expression is enhanced by CD28 costimulation and regulates differentiation of CD4(+) T cells. J Immunol. 2000 Nov. 1;165(9):5035-40.

10. Coyle A J, Lehar S, Lloyd C, Tian J, Delaney T, Manning S, Nguyen T, Burwell T, Schneider H, Gonzalo J A, Gosselin M, Owen L R, Rudd C E, Gutierrez-Ramos J C. The CD28-related molecule ICOS is required for effective T cell-dependent immune responses. Immunity. 2000 July;13(1):95-105.

11. David P. Harris, Laura Haynes, Peter C. Sayles, Debra K Duso, Sheri M. Eaton, Nancy M. Lepak, Lawrence L. Johnson, Susan L. Swain, Frances E. Lund. Reciprocal regulation of polarized cytokine production by effector B and T cells. Nature Immunology. 2000 December;1(6): 475-482.

12. Tripp C S, Wolf S F, Unanue E R. Interleukin 12 and tumor necrosis factor alpha are costimulators of interferon gamma production by natural killer cells in severe combined immunodeficiency mice with listeriosis, and interleukin 10 is a physiologic antagonist. Proc Natl Acad Sci USA. 1993 Apr. 15;90(8):3725-9.

13. Higuchi, R., Dollinger, G., Walsh, P. S. and Griffith, R. (1992) Simultaneous amplification and detection of specific DNA sequences. BioTechnology 10:413-417.

14. Higuchi, R., Fockler, C., Dollinger, G. and Watson, R. (1993) Kinetic PCR analysis: real-time monitoring of DNA amplification reactions. BioTechnology 11:1026-1030.

15. T. B. Morrison, J. J. Weis & C. T. Wittwer (1998) Quantification of low-copy transcripts by continuous SYBR Green I monitoring during amplification. Biotechniques 24:954-962.

16. Adams, M. D., Kerlavage, A. R., Fields, C. & Venter, C. (1993) 3,400 new expressed sequence tags identify diversity of transcripts in human brain. Nature Genet. 4:256-265.

17. Adams, M. D., et al. (1995) Initial assessment of human gene diversity and expression patterns based upon 83 million nucleotides of cDNA sequence. Nature 377 supp:3-174.

18. Liew, C. C., Hwang, D. M., Fung, Y. W., Laurenson, C., Cukerman, E., Tsui, S. & Lee, C. Y. (1994) A catalog of genes in the cardiovascular system as identified by expressed sequence tags. Proc. Natl. Acad. Sci. USA 91:10145-10649.

1. An isolated polynucleotide being selected from the group consisting of:
a) a polynucleotide encoding a B7-H2 V polypeptide comprising an amino acid sequence selected form the group consisting of:
amino acid sequences which are at least about 90% identical to the amino acid sequence shown in SEQ ID NO: 3;
amino acid sequences which are at least about 90% identical to the amino acid sequence shown in SEQ ID NO:4;
the amino acid sequence shown in SEQ ID NO:3 and the amino acid sequence shown in SEQ ID NO: 4.
b) a polynucleotide comprising the sequence of SEQ ID NO:1 or SEQ ID NO:2;
c) a polynucleotide which hybridizes under stringent conditions to a polynucleotide specified in (a) and (b) and encodes a B7-H2 V polypeptide;
d) a polynucleotide the sequence of which deviates from the polynucleotide sequences specified in (a) to (c) due to the degeneration of the genetic code and encodes a B7-H2 V polypeptide; and
e) a polynucleotide which represents a fragment, derivative or allelic variation of a polynucleotide sequence specified in (a) to (d) and encodes a B7-H2 V polypeptide.

2. An expression vector containing any polynucleotide of claim 1.

3. A host cell containing the expression vector of claim 2.

4. A substantially purified B7-H2 V polypeptide encoded by a polynucleotide of claim 1.

5. A method for producing a B7-H2 V polypeptide, wherein the method comprises the steps of:
a) culturing the host cell of claim 3 under conditions suitable for the expression of the B7-H2 V polypeptide; and
b) recovering the B7-H2 V polypeptide from the host cell culture.

6. A method for the detection of polynucleotides encoding a B7-H2 V polypeptide in a biological sample comprising the steps of:
a) hybrizing a polynucleotide of claim 1 to nucleic acid material of a biological sample, thereby forming a hybridization complex; and
b) detecting said hybridization complex; wherein the presence of said
complex correlates with the presence of a polynucleotide encoding the B7-H2 in said biological sample.

7. The method of claim 6, wherein before hybridization, the nucleic acid material of the biological sample is amplified.

8. A method for the detection of a polynucleotide of claim 1 or a B7-H2 V polypeptide of claim 4 comprising the steps of contacting a biological sample with a reagent which specifically interacts with the polynucleotide of the B7-H2 V polypeptide.

9. A diagnostic kit for conducting the method of any one of claim 6 to 8.

10. A method of screening for agents which decrease the activity of B7-H2 V, comprising the steps of:
contacting a test compound with any B7-H2 V polypeptide encoded by any polynucleotide of claim 1;
and detecting binding of the test compound to the B7-H2 V polypeptide, wherein a test compound which binds to the polypeptide is identified as a potential therapeutic agent for decreasing the activity of B7-H2 V.

11. A method of screening for agents which regulate the activity of B7-H2 V, comprising the steps of:
contacting a test compound with any B7-H2 V polypeptide encoded by any polynucleotide of claim 1; and
detecting B7-H2 V activity of the polypeptide, wherein a test compound which increases the B7-H2 V activity is identified as a potential therapeutic agent for increasing the activity of B7-H2 V, and wherein a test compound which decreases the B7-H2 V activity of the polypeptide is identified as a potential therapeutic agent for decreasing the activity of B7-H2 V.

12. A method of screening for agents which decrease the activity of B7-H2 V, comprising the steps of:
contacting a test compound with any B7-H2 V polynucleotide of claim 1; and
detecting binding of the test compound to the polynucleotide, wherein a test compound which binds to the polynucleotide is identified as a potential therapeutic agent for decreasing the activity of B7-H2 V.

13. A method of reducing the activity of B7-H2 V, comprising the steps of:
contacting a cell with a reagent which specifically binds to any polynucleotide of claim 1; or
any polypeptide of claim 4, whereby the activity of B7-H2 V is reduced.

14. A reagent that modulates the activity of B7-H2 V polypeptide or a polynucleotide wherein said reagent is identified by the method of any of the claim 10 to 12.

15. A pharmaceutical composition, comprising the reagent of claim 14 and a pharmaceutically acceptable carrier.

16. Use of the reagent of claim 14 in the preparation of a medicament for modulating the activity of B7-H2 V in a disease.

17. Use of claim 16 wherein the disease is an infectious disease, asthma or an allergic or inflammatory disease.

18. A method of screening for agents which can regulate the activity of B7-H2 V protein, comprising the steps of:
contacting a test compound with a polypeptide comprising an amino acid sequence which is at least about 90% identical to the amino acid sequence shown in SEQ ID NO: 3 or 4; or the sequence shown in SEQ ID NO: 3 or 4;
and detecting binding of the test compound to the polypeptide, wherein a test compound which binds to the polypeptide is identified as a potential agent for regulating activity of the B7-H2 V protein.

19. A method of claim 18 wherein the step of contacting is in a cell.

20. The method of claim 18 wherein the cell is in vitro.

21. The method of claim 18 wherein the step of contacting is in a cell-free system.

22. The method of claim 18 wherein the polypeptide comprises a detectable label.

23. The method of claim 18 wherein the test compound comprises a detectable label.

24. The method of claim 18 wherein the test compound displaces a labeled ligand which is bound to the polypeptide.

25. The method of claim 18 wherein the polypeptide is bound to a solid support.

26. The method of claim 18 wherein the test compound is bound to a solid support.

27. A method of screening for agents which regulate the activity of B7-H2 V protein, comprising the steps of:
contacting a test compound with a polypeptide comprising an amino acid sequence which is at least about 90% identical to the amino acid sequence shown in SEQ ID NO: 3 or 4; or the sequence shown in SEQ ID NO: 3 or 4; and
detecting an activity of the polypeptide, wherein a test compound which increases the activity of the polypeptide is identified as a potential agent for increasing the activity of the human B7-H2 V protein, and wherein a test compound which decreases the activity of the polypeptide is identified as a potential agent for decreasing the activity of the human B7-H2 V protein.

28. The method of claim 27 wherein the step of contacting is in a cell.

29. The method of claim 27 wherein the cell is in vitro.

30. The method of claim 27 wherein the step of contacting is in a cell-free system.

31. A method of screening for agents which regulate B7-H2 V protein, comprising the steps of:
contacting a test compound with a product encoded by a polynucleotide which comprises the nucleotide sequence shown in SEQ ID NO:1 or 2; and
detecting binding of the test compound to the product, wherein a test compound which binds to the product is identified as a potential agent for regulating the activity of human B7-H2 V protein.

32. The method of claim 31 wherein the product is a polypeptide.

33. The method of claim 31 wherein the product is RNA.

34. A method of reducing activity of a human B7-H2 V protein, comprising the step of:
contacting a cell with a reagent which specifically binds to a product encoded by a polynucleotide comprising the nucleotide sequence shown in SEQ ID NO:1 or 2, whereby the activity of a human B7-H2 V protein is reduced.

35. The method of claim 34 wherein the product is a polypeptide.

36. The method of claim 35 wherein the reagent is an antibody.

37. The method of claim 35 wherein the product is RNA.

38. The method of claim 34 wherein the reagent is an antisense oligonucleotide.

39. The method of claim 34 wherein the reagent is a ribozyme.

40. The method of claim 34 wherein the cell is in vitro.

41. The method of claim 34 wherein the cell is in vivo.

42. A pharmaceutical composition, comprising:
a reagent which specifically binds to a polypeptide comprising the amino acid sequence shown in SEQ ID NO:3 or 4; and
a pharmaceutically acceptable carrier.

43. The pharmaceutical composition of claim 42 wherein the reagent is an antibody.

44. A pharmaceutical composition, comprising:
a reagent which specifically binds to a product of a polynucleotide comprising the nucleotide sequence shown in SEQ ID NO:1 or 2; and
a pharmaceutically acceptable carrier.

45. The pharmaceutical composition of claim 44 wherein the reagent is a ribozyme.

46. The pharmaceutical composition of claim 44 wherein the reagent is an antisense oligonucleotide.

47. The pharmaceutical composition of claim 44 wherein the reagent is an antibody.

48. A method of treating B7-H2 V dysfunction related disease, wherein the disease is selected from an infectious disease, asthma, or an allergic or inflammatory disease comprising the step of:
administering to a patient in need thereof a therapeutically effective dose of a reagent that regulates the function of human B7-H2 V protein, whereby symptoms of the B7-H2 V dysfunction related disease are ameliorated.

49. The method of claim 48 wherein the reagent is identified by the method of claim 18.

50. The method of claim 48 wherein the reagent is identified by the method of claim 27.

51. The method of claim 48 wherein the reagent is identified by the method of claim 31.


SEQUENCE LISTING (8 sequences)

SEQ ID NO: 1 - DNA, 1064 bp, Homo sapiens
cgaggttgctcctctccgaggtctcccgcggcccaagttctccgcgccccgaggtctccgcgccccgaggtctccgcggcccgaggtctccgcccgcaccatgcggctgggcagtcctggactgctcttcctgctcttcagcagccttcgagctgatactcaggagaaggaagtcagagcgatggtaggcagcgacgtggagctcagctgcgcttgccctgaaggaagccgttttgatttaaatgatgtttacgtatattggcaaaccagtgagtcgaaaaccgtggtgacctaccacatcccacagaacagctccttggaaaacgtggacagccgctaccggaaccgagccctgatgtcaccggccggcatgctgcggggcgacttctccctgcgcttgttcaacgtcaccccccaggacgagcagaagtttcactgcctggtgttgagccaatccctgggattccaggaggttttgagcattgaggttacactgcatgtggcagcaaacttcagcgtgcccgtcgtcagcgccccccacagcccctcccaggatgagctcaccttcacgtgtacatccataaacggctaccccaggcccaacgtgtactggatcaataagacggacaacagcctgctggaccaggctctgcagaatgacaccgtcttcttgaacatgcggggcttgtatgacgtggtcagcgtgctgaggatcgcacggacccccagcgtgaacattggctgctgcatagagaacgtgcttctgcagcagaacctgactgtcggcagccagacaggaaatgacatcggagagagagacaagatcacagagaatccagtcagtaccggcgagaaaaacgcggccacgtggagcatcctggctgtcctgtgcctgcttgtggtcgtggcggtggccataggctgggtgtgcagggaccgatgcctccaacacagctatgcaggtgcctgggctgtgagtccggagacagagctcactgttccaggagcaacatagatgtggattcctgtccaatttgggaaaaatgtccacacacggtcaccca

SEQ ID NO: 2 - DNA, 1571 bp, Homo sapiens
cgaggttgctcctctccgaggtctcccgcggcccaagttctccgcgccccgaggtctccgcgccccgaggtctccgcggcccgaggtctccgcccgcaccatgcggctgggcagtcctggactgctcttcctgctcttcagcagccttcgagctgatactcaggagaaggaagtcagagcgatggtaggcagcgacgtggagctcagctgcgcttgccctgaaggaagccgttttgatttaaatgatgtttacgtatattggcaaaccagtgagtcgaaaaccgtggtgacctaccacatcccacagaacagctccttggaaaacgtggacagccgctaccggaaccgagccctgatgtcaccggccggcatgctgcggggcgacttctccctgcgcttgttcaacgtcaccccccaggacgagcagaagtttcactgcctggtgttgagccaatccctgggattccaggaggttttgagcattgaggttacactgcatgtggcagcaaacttcagcgtgcccgtcgtcagcgccccccacagcccctcccaggatgagctcaccttcacgtgtacatccataaacggctaccccaggcccaacgtgtactggatcaataagacggacaacagcctgctggaccaggctctgcagaatgacaccgtcttcttgaacatgcggggcttgtatgacgtggtcagcgtgctgaggatcgcacggacccccagcgtgaacattggctgctgcatagagaacgtgcttctgcagcagaacctgactgtcggcagccagacaggaaatgacatcggagagagagacaagatcacagagaatccagtcagtaccggcgagaaaaacgcggccacgtggagcatcctggctgtcctgtgcctgcttgtggtcgtggcggtggccataggctgggtgtgcagggaccgatgcctccaacacagctatgcaggtgcctgggctgtgagtccggagacagagctcactggtgagtttgccgtgggaagcagcaggttctggggggcccaggggaggcttggctgccagctgtctttcagagtttcaaaaaactttcaaaaggcaaaagtcccttgccttgaacaactgttgttcctggagacgcagcgaagccctcgatggtgcgcatggcatttcctgcagcctccccttggcatgggatggcatcctggtgtgcactttgtcacactgcgatgggattttcccaacatgcacagaagcagagagacgagtgctagacccccgcgctccccagtgcccagccccaaccagggtgtccagggcgggtccaggcaccggcgcccagcccccatggggtgtccggagtgggtccaggcaccggcgcccagcccccgtggggtgtccagggcgggtccaggcaccggcgcccagcccctgtggggtgtccggagcgggtccgggcaccgccagcttctctctgtggcagccactcctgcagctctcgtttgcccctcagttccaggagcaacatagatgtggattcctgtccaatttgggaaaaatgtccacacacggtcaccca

SEQ ID NO: 3 - protein, 304 aa, Homo sapiens
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSIEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTVPGAT

SEQ ID NO: 4 - protein, 473 aa, Homo sapiens
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSIEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGEFAVGSSRFWGAQGRLGCQLSFRVSKNFQKAKVPCLEQLLFLETQRSPRWCAWHFLQPPLGMGWHPGVHFVTLRWDFPNMHRSRETSARPPRSPVPSPNQGVQGGSRHRRPAPMGCPEWVQAPAPSPRGVSRAGPGTGAQPLWGVRSGSGHRQLLSVAATPAALVCPSVPGAT

SEQ ID NO: 5 - DNA, 24 bp, Homo sapiens
  misc_feature (1)..(24): Primer  K653-L2
cgaggttgctcctctccgaggtct

SEQ ID NO: 6 - DNA, 24 bp, Homo sapiens
  misc_feature (1)..(24): Primer  K653-R6
tgggtgaccgtgtgtggacatttt

SEQ ID NO: 7 - DNA, 24 bp, Homo sapiens
  misc_feature (1)..(24): Primer  K653-L5
aggttacactgcatgtggcagcaa

SEQ ID NO: 8 - DNA, 24 bp, Homo sapiens
  misc_feature (1)..(24): Primer  K653-R8
cgtttttctcgccggtactgactg